(19) United States
(12) Patent Application Publication
(10) Pub. No.: US 2018/0291353 A1
(43) Pub. Date: Oct. 11, 2018

(54) MONOFUNCTIONAL ALDEHYDE AND ALCOHOL DEHYDROGENASES FOR PRODUCTION OF FUELS AND COMMODITY CHEMICALS

(71) Applicants: The Regents of the University of California, Oakland, CA (US); Calysta, Inc., Menlo Park, CA (US)

(72) Inventors: Michelle C.Y. CHANG, Berkeley, CA (US); Matthew DAVIS, Berkeley, CA (US); Josh SILVERMAN, Menlo Park, CA (US); Drew REGITSKY, Menlo Park, CA (US)

(73) Assignees: The Regents of the University of California, Oakland, CA (US); Calysta, Inc., Menlo Park, CA (US)

(21) Appl. No.: 15/575,709

(22) PCT Filed: May 27, 2016
(86) PCT No.: PCT/US2016/034798

§ 371 Date: Nov. 20, 2017

Related U.S. Application Data
(60) Provisional application No. 62/167,841, filed on May 28, 2015.

(51) Int. Cl. C12N 9/04; C12N 9/02; C12P 7/16; C12P 7/26; C12P 7/18; C12P 7/04; C12N 9/88

(52) U.S. Cl. CPC C12N 9/0006; C12Y 101/01001; C12Y 102/0101; C12N 9/0008; C12Y 103/01038; C12N 9/88; C12P 7/16; C12P 7/26; C12P 7/18; C12P 7/04; C12Y 402/01017; C12N 9/001

(57) ABSTRACT

The present disclosure relates generally to the production of alcohols, and more specifically to biological platforms for the production of alcohols using monofunctional aldehyde dehydrogenases and monofunctional alcohol dehydrogenases.

### CROSS-REFERENCE TO RELATED APPLICATION

This application claims the benefit of U.S. Provisional Application No. 62/167,841, filed May 28, 2015, which is hereby incorporated herein by reference in its entirety.

### STATEMENT REGARDING FEDERALLY-SPONSORED RESEARCH

This invention was made with government support under Grant No. 30221-10805-CCMCC awarded by the National Science Foundation. The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 416272010340SeqList.txt, date recorded: May 26, 2016, size: 1,285 KB).

### FIELD

The present disclosure relates generally to the production of alcohols, and more specifically to biological platforms for the production of alcohols using monofunctional aldehyde dehydrogenases and monofunctional alcohol dehydrogenases.

### BACKGROUND

The rise in global energy usage, together with the disappearance of fossil fuel reserves, has highlighted the importance of developing technologies to harness new and renewable energy sources. In addition to sustainability, climate change is another major issue that has driven the search for clean, carbon-neutral fuels and commodity chemicals. In an effort to meet these goals, chemicals derived from plant biomass are being explored as potential substitutes. In this approach, abundant and renewable plant material is harvested as a feedstock for microbial fermentation after biomass pretreatment and processing. The major biofuel in use today is ethanol, but ethanol has major shortcomings including the low energy return compared to gasoline, high vaporizability, as well as miscibility with water.

In view of these facts and the growing global demand in biofuels, a significant need exists for improved biofuels and methods for biofuel synthesis, particularly for biofuels that exhibit improved characteristics over ethanol.

### BRIEF SUMMARY

In one aspect, the present disclosure relates to recombinant host cell that facilitates the production of an alcohol from an acyl-CoA, where the host cell includes: a) a first nucleic acid which encodes a polypeptide involved in the stepwise conversion of an acyl-CoA to a substrate for a monofunctional aldehyde dehydrogenase; b) a second nucleic acid which encodes a monofunctional aldehyde dehydrogenase; and c) a third nucleic acid which encodes a monofunctional alcohol dehydrogenase; where at least one nucleic acid selected from the group of the first nucleic acid, the second nucleic acid, and the third nucleic acid is a recombinant nucleic acid. In some embodiments, the host cell is E. coli. In some embodiments that may be combined with any of the preceding embodiments, at least two nucleic acids selected from the group of the first nucleic acid, the second nucleic acid, and the third nucleic acid are separate nucleic acids. In some embodiments that may be combined with any of the preceding embodiments, the recombinant nucleic acid encodes a polypeptide selected from the group of an acetoacetyl-CoA thiolase, a 3-hydroxybutyryl-CoA dehydrogenase, a crotonase, a trans-enoyl-CoA reductase, a monofunctional aldehyde dehydrogenase, and a monofunctional alcohol dehydrogenase. In some embodiments, the acetoacetyl-CoA thiolase has at least 80% amino acid identity to SEQ ID NO: 33, the 3-hydroxybutyryl-CoA dehydrogenase has at least 80% amino acid identity to SEQ ID NO: 34, the crotonase has at least 80% amino acid identity to SEQ ID NO: 36, and the trans-enoyl-CoA reductase has at least 80% amino acid identity to SEQ ID NO: 37. In some embodiments that may be combined with any of the preceding embodiments, the monofunctional aldehyde dehydrogenase has at least 80% amino acid identity to SEQ ID NO: 16 and the monofunctional alcohol dehydrogenase has at least 80% amino acid identity to SEQ ID NO: 17. In some embodiments that may be combined with any of the preceding embodiments, the acyl-CoA is acetyl-CoA. In some embodiments that may be combined with any of the preceding embodiments, the alcohol is selected from the group of n-butanol, crotyl alcohol, 1,3-butanediol, and 4-hydroxy-2-butanone. In some embodiments that may be combined with any of the preceding embodiments, the host cell exhibits reduced activity of one or more polypeptides selected from the group of adhE, ldhA, ack-pta, poxB, and frdBC, or homologs thereof, as compared to a corresponding control cell. In some embodiments, the host cell includes knockout mutations in adhE, ldhA, ack-pta, poxB, and frdBC, or homologs thereof. In some embodiments that may be combined with any of the preceding embodiments, the host cell further includes a monofunctional secondary alcohol dehydrogenase. In some embodiments, the monofunctional secondary aldehyde dehydrogenase has at least 80% amino acid identity to SEQ ID NO: 250.

In another aspect, the present disclosure relates to a recombinant host cell for the production of n-butanol, the host cell including: a) a nucleic acid encoding an acetoacetyl-CoA thiolase capable of catalyzing the conversion of acetyl-CoA to acetoacetyl-CoA; b) a nucleic acid encoding a 3-hydroxybutyryl-CoA dehydrogenase capable of catalyzing the conversion of acetoacetyl-CoA to 3-hydroxybutyryl-CoA; c) a nucleic acid encoding a crotonase capable of catalyzing the conversion of 3-hydroxybutyryl-CoA to crotonyl-CoA; d) a nucleic acid encoding a trans-enoyl-CoA reductase capable of catalyzing the conversion of crotonyl-CoA to butyryl-CoA; e) a nucleic acid encoding a monofunctional aldehyde dehydrogenase capable of catalyzing the conversion of butyryl-CoA to butyraldehyde; and f) a nucleic acid encoding a monofunctional alcohol dehydrogenase capable of catalyzing the conversion of butyraldehyde to n-butanol, where one or more of the nucleic acids are recombinant, and where the host cell is capable of producing at least 10-fold more n-butanol than ethanol.

In another aspect, the present disclosure relates to a recombinant host cell for the production of crotyl alcohol, the host cell including: a) a nucleic acid encoding an acetoacetyl-CoA thiolase capable of catalyzing the conversion of acetyl-CoA to acetoacetyl-CoA; b) a nucleic acid encoding a 3-hydroxybutyryl-CoA dehydrogenase capable of catalyzing the conversion of acetoacetyl-CoA to 3-hydroxybutyryl-CoA; c) a nucleic acid encoding a crotonase capable of catalyzing the conversion of 3-hydroxybutyryl-CoA to crotonyl-CoA; d) a nucleic acid encoding a monofunctional aldehyde dehydrogenase capable of catalyzing the conversion of crotonyl-CoA to crotonaldehyde; and e) a nucleic acid encoding a monofunctional alcohol dehydrogenase capable of catalyzing the conversion of crotonaldehyde to crotyl alcohol, where one or more of the nucleic acids are recombinant.

In another aspect, the present disclosure relates to a recombinant host cell for the production of 1,3-butanediol, the host cell including: a) a nucleic acid encoding an acetoacetyl-CoA thiolase capable of catalyzing the conversion of acetyl-CoA to acetoacetyl-CoA; b) a nucleic acid encoding a 3-hydroxybutyryl-CoA dehydrogenase capable of catalyzing the conversion of acetoacetyl-CoA to 3-hydroxybutyryl-CoA; c) a nucleic acid encoding a monofunctional aldehyde dehydrogenase capable of catalyzing the conversion of 3-hydroxybutyryl-CoA to 3-hydroxybutyraldehyde; and d) a nucleic acid encoding a monofunctional alcohol dehydrogenase capable of catalyzing the conversion of 3-hydroxybutyraldehyde to 1,3-butanediol, where one or more of the nucleic acids are recombinant.

In another aspect, the present disclosure relates to a recombinant host cell for the production of 4-hydroxy-2-butanone, the host cell including: a) a nucleic acid encoding an acetoacetyl-CoA thiolase capable of catalyzing the conversion of acetyl-CoA to acetoacetyl-CoA; b) a nucleic acid encoding a monofunctional aldehyde dehydrogenase; and c) a nucleic acid encoding a monofunctional alcohol dehydrogenase, where one or more of the nucleic acids are recombinant.

In another aspect, the present disclosure relates to a recombinant host cell for the production of one or more C4 alcohols, the host cell including: a) a nucleic acid encoding an acetoacetyl-CoA thiolase; b) a nucleic acid encoding a 3-hydroxybutyryl-CoA dehydrogenase; c) a nucleic acid encoding a crotonase; d) a nucleic acid encoding a trans-enoyl-CoA reductase; e) a nucleic acid encoding a monofunctional aldehyde dehydrogenase; and f) a nucleic acid encoding a monofunctional alcohol dehydrogenase, where one or more of the nucleic acids are recombinant, and where the host cell is capable of producing a C4 alcohol at concentrations that are at least 10-fold higher than the concentration of ethanol produced by the host cell. In some embodiments, the C4 alcohol is selected from the group of n-butanol, crotyl alcohol, 1,3-butanediol, and 4-hydroxy-2-butanone.

In another aspect, the present disclosure relates to a method of producing an alcohol from an acyl-CoA, the method including: a) providing the recombinant host cell of any one of the preceding embodiments; and b) culturing the recombinant host cell in a culture medium including a suitable carbon source such that the host cell produces an alcohol. In some embodiments, the method further includes a step of substantially purifying the alcohol from the culture medium.

In another aspect, the present disclosure relates to a method of producing n-butanol, the method including: a) providing the recombinant host cell that produces n-butanol; and b) culturing the recombinant host cell in a culture medium including a suitable carbon source such that the host cell produces n-butanol, where the host cell produces at least 10-fold more n-butanol than ethanol. In some embodiments, the method further includes a step of substantially purifying n-butanol from the culture medium.

In another aspect, the present disclosure relates to a method of producing crotyl alcohol, the method including: a) providing the recombinant host cell that produces crotyl alcohol; and b) culturing the recombinant host cell in a culture medium including a suitable carbon source such that the host cell produces crotyl alcohol. In some embodiments, the method further includes a step of substantially purifying crotyl alcohol from the culture medium.

In another aspect, the present disclosure relates to a method of producing 1,3-butanediol, the method including: a) providing the recombinant host cell that produces 1,3-butanediol; and b) culturing the recombinant host cell in a culture medium including a suitable carbon source such that the host cell produces 1,3-butanediol. In some embodiments, the method further includes a step of substantially purifying 1,3-butanediol from the culture medium.

In another aspect, the present disclosure relates to a method of producing 4-hydroxy-2-butanone, the method including: a) providing the recombinant host cell that produces 4-hydroxy-2-butanone; and b) culturing the recombinant host cell in a culture medium including a suitable carbon source such that the host cell produces 4-hydroxy-2-butanone. In some embodiments, the method further includes a step of substantially purifying 4-hydroxy-2-butanone from the culture medium.

In another aspect, the present disclosure relates to a method of producing one or more C4 alcohols, the method including: a) providing the recombinant host cell that produces a C4 alcohol; and b) culturing the recombinant host cell in a culture medium including a suitable carbon source such that the host cell produces a C4 alcohol, where the host cell produces the C4 alcohol at concentrations that are at least 10-fold higher than the concentration of ethanol produced by the host cell. In some embodiments, the method further includes a step of substantially purifying the C4 alcohol from the culture medium. In some embodiments that may be combined with any of the preceding embodiments, the C4 alcohol is selected from the group of n-butanol, crotyl alcohol, 1,3-butanediol, and 4-hydroxy-2-butanone.

In another aspect, the present disclosure relates to a recombinant polypeptide including an amino acid sequence selected from the group of SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, and SEQ ID NO: 154.

In another aspect, the present disclosure relates to a recombinant nucleic acid including a nucleotide sequence selected from the group of SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, and SEQ ID NO: 249.

In another aspect, the present disclosure relates to a host cell including a recombinant polypeptide of any of the preceding embodiments, or a host cell including a recombinant nucleic acid of any of the preceding embodiments.

### DESCRIPTION OF THE FIGURES

FIG. 1 illustrates a model demonstrating channeling of the volatile aldehyde intermediate in bifunctional AdhE enzymes. Both activities (aldehyde dehydrogenase activity and alcohol dehydrogenase activity) are contained on the same polypeptide and it is believed that this organization helps to channel the acyl-CoA substrate to the final alcohol product.

FIG. 2A-FIG. 2B illustrate that AdhE2 mutants do not produce significantly more butanol than wild-type (WT) AdhE2 in conjunction with lower ethanol production. The fuel titer of butanol in E. coli cells expressing the biosynthetic pathway shown in FIG. 6A with various particular AdhE2 variants incorporating amino acid substitutions from the AdhE2 superfamily is shown. The x-axis labels indicate the particular AdhE2 variant being expressed in the E. coli, with wild-type AdhE2 also shown for comparison. In each figure, E. coli DH1 was transformed with pT5T33-phaA.HBD-crt and pCDF3-ter-AdhE2 variant as indicated. FIG. 2A illustrates the butanol production for AdhE2 variants #76930 to #76978. FIG. 2B illustrates the butanol production for AdhE2 variants #76979 to #77025, and also includes WT AdhE2 (GI 499193180). AdhE2 and variants thereof shown in FIG. 2A and FIG. 2B include SEQ ID NOs: 60-154.

FIG. 3 illustrates that bifunctional AdhE2 homologs do not have improved specificity for C4 substrates. The fuel titer of either butanol or ethanol in E. coli cells expressing the biosynthetic pathway shown in FIG. 6A with various particular AdhE2 homologs is shown. Specifically, E. coli DH1 was transformed with pT5T33-phaA.HBD-crt and pCDF3-ter-AdhE2 homolog as indicate or with WT AdhE2. The x-axis labels indicate the particular AdhE2 homolog being expressed (GI accession) in the E. coli, with wild-type AdhE2 also shown for comparison. AdhE2 homologs shown include SEQ ID NOs: 42-59.

FIG. 4A-FIG. 4B illustrate in vitro preparation and characterization of aldehyde dehydrogenase 16 (ALDH16). As an example of a monofunctional aldehyde dehydrogenase useful for the production of butanol, ALDH16 was purified and characterized in vitro. This characterization revealed ALDH16 to exist as a tetramer and exhibit 73-fold specificity for butyryl-CoA vs acetyl-CoA. FIG. 4A illustrates SDS-PAGE gel of HisTEV-ALDH16 purification. FIG. 4B illustrates a size-exclusion chromatogram of GA-ALDH16.

FIG. 5 illustrates genetically engineered improvement of a butanol production pathway composed of monofunctional aldehyde and alcohol dehydrogenases. The fuel titer of either butanol or ethanol in E. coli cells expressing the biosynthetic pathway shown in FIG. 6A with various particular alcohol and/or aldehyde dehydrogenases is shown. Specifically, E. coli MC1.24 (quintuple KO) was transformed with pT533-phaA.HBD, pBBR2-aceE.F.lpd and 1: pCWO.trc-ter-adhE2, 2: pCDF3-aldh16, 3: pCWori-ter-aldh16.CaADH, or 4: pCWO.trc-ter-aldh16.CaADH as indicated.

FIG. 6A-FIG. 6C illustrate exemplary biosynthetic pathways for butanol (FIG. 6A), crotyl alcohol (FIG. 6B), and 1,3-butanediol (FIG. 6C) production.

FIG. 7 illustrates butanol, 1,3-butanediol, and crotyl alcohol production in E. coli genetically engineered to express one of the biosynthetic pathways presented in FIG. 6A-FIG. 6C. The genetically modified cells express either the biosynthetic pathway for butanol (FIG. 6A), crotyl alcohol (FIG. 6B), or 1,3-butanediol (FIG. 6C) production. Each group of bars on the X-axis indicates a different E. coli cell line expressing a particular aldehyde dehydrogenase (ALDH) in the specific biosynthetic pathway as described above. Specifically, E. coli MC1.24 (quintuple KO) was transformed with pT5T33-phaA.HBD-crt and pCDF3-ter-aldh 1-16 as indicated for butanol production, pT533-phaA.HBD and pCDF3-aldh 1-16 as indicated for 1,3-butanediol production, or pT5T33-phaA.HBD-crt and pCDF3-aldh 1-16 as indicated for crotyl alcohol production. Each E. coli line contained only one of the recombinant aldh genes 1-16.

FIG. 8 illustrates a sequence similarity network of alcohol dehydrogenases and 1,3-propanediol dehydrogenases. The alcohol dehydrogenase sequence family (EC 1.1.1.1) was downloaded from Pfam (PF00465) and filtered using CD-HIT (cd-hit.org) to remove sequences of greater than 80% identity. The remaining sequences were compared with all-vs-all protein BLAST and the results were imported to Cytoscape (cytoscape.org) to visualize clusters of related protein sequences. Protein sequences are represented as nodes, which are connected by edges if the BLAST e-value between two proteins is above an arbitrary cutoff. An e-value cutoff of e-100 was chosen to separate various classes of alcohol dehydrogenases (e.g. butanol dehydrogenases and 1,3-propanediol dehydrogenases) to identify potential alcohol dehydrogenases for production of 1,3-butanediol. Alcohol dehydrogenases 1-16 were then randomly selected from the resulting clusters.

FIG. 9 illustrates a gas chromatogram and EI mass spectrum of 1,3-butanediol produced by engineered E. coli. Culture supernatant from E. coli strains harboring 1,3-butanediol production pathways was analyzed by GC-MS. Retention time and fragmentation pattern agree with a commercial authentic standard (Sigma).

FIG. 10 illustrates the screening of alcohol dehydrogenases useful for production of 1,3-butanediol. Alcohol dehydrogenases 1-16 (listed by UniProt ID) were identified bioinformatically (See FIG. 8) and cloned into plasmid pCWO.trc-aldh16 to generate pairs of aldehyde and alcohol dehydrogenases useful for production of 1,3-butanediol. E. coli MC1.24 (DH1 ΔadhE ΔldhA Δack-pta ΔpoxB ΔfrdBC) was transformed with pT533-phaA.phaB and pCWO.trc-aldh16.adh 1-16 as indicated (by UniProt ID) and cultured anaerobically for 3 days. Culture supernatant was harvested and 1,3-butanediol titers were quantified by GC-MS. Each E. coli line contained only one of the recombinant adh genes 1-16.

FIG. 11 illustrates 1,3-butanediol and 4-hydroxy-2-butanone production using various combinations of monofunctional aldehyde and alcohol dehydrogenases. E. coli MC1.24 (DH1 ΔadhE ΔldhA Δack-pta ΔpoxB ΔfrdBC) was transformed with pT533-phaA.phaB and engineered to express the specific aldh and adh as indicated (pCWO.trc-aldh.adh). Cells were cultured anaerobically for 3 days. Culture supernatant was harvested and 1,3-butanediol and 4-hydroxy-2-butanone titers were quantified by GC-MS.

FIG. 12A-FIG. 12C illustrate exemplary variant pathways for 4-hydroxy-2-butanone production (FIG. 12A), 1,3-butanediol production (FIG. 12C), or both 4-hydroxy-2-butanone and 1,3-butanediol production (FIG. 12B).

FIG. 13 illustrates the production titers of 4-hydroxy-2-butanone and/or 1,3-butanediol by expressing the pathway described in FIG. 12A, FIG. 12B, or FIG. 12C in E. coli MC1.24.

FIG. 14 illustrates a size-exclusion chromatogram of GA-ALDH3.

FIG. 15 illustrates an exemplary variant pathway for control of pathway side-products resulting from a promiscuous ALDH and ADH pair by expression of a secondary alcohol dehydrogenase. Expression of a secondary alcohol dehydrogenase can reduce any accumulated 4-hydroxy-2-butanone (hydroxybutanone) to 1,3-butanediol (butanediol).

FIG. 16 illustrates the results of a screen of secondary alcohol dehydrogenases when expressed in the pathway described in FIG. 15. The production titers of 4-hydroxy-2-butanone (hydroxybutanone) and 1,3-butanediol (butanediol) when the illustrated secondary alcohol dehydrogenases are expressed in the pathway illustrated in FIG. 15 are shown. Data are mean±s.d. (n=3).

FIG. 17 illustrates how expression of specific proteins in the pathway described in FIG. 15 can control butanediol:hydroxybutanone ratios through pathway design. The production titers of 4-hydroxy-2-butanone (hydroxybutanone) and 1,3-butanediol (butanediol) when the illustrated proteins are expressed in the pathway illustrated in FIG. 15 are shown. Data are mean±s.d. (n=3).

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates generally to the production of alcohols, and more specifically to biological platforms for the production of alcohols using monofunctional aldehyde dehydrogenases and monofunctional alcohol dehydrogenases.

The present disclosure is based, at least in part, on Applicants discovery that monofunctional aldehyde dehydrogenases and monofunctional alcohol dehydrogenases can be used to produce various alcohols in host cells. Previously constructed biosynthetic pathways for the production of alcohols have used bifunctional aldehyde/alcohol dehydrogenases (i.e. a single enzyme that has both aldehyde dehydrogenase activity and alcohol dehydrogenase activity). Monofunctional aldehyde dehydrogenases and monofunctional alcohol dehydrogenases may differ from bifunctional aldehyde/alcohol dehydrogenases in that the monofunctional enzymes do not have both aldehyde dehydrogenase activity and alcohol dehydrogenase activity, but instead have only one of the respective aldehyde dehydrogenase or alcohol dehydrogenase enzymatic activities. A monofunctional aldehyde dehydrogenase may include, for example, an enzyme that has aldehyde dehydrogenase activity and no detectable alcohol dehydrogenase activity. A monofunctional alcohol dehydrogenase may include, for example, an enzyme that has alcohol dehydrogenase activity and no detectable aldehyde dehydrogenase activity. In host cells that contain a nucleic acid that encodes a monofunctional aldehyde dehydrogenase and a nucleic acid that encodes a monofunctional alcohol dehydrogenase, the monofunctional aldehyde dehydrogenase and the monofunctional alcohol dehydrogenase are encoded as separate polypeptides.

Without wishing to be bound by theory, it was thought that the mechanism for a bifunctional aldehyde/alcohol dehydrogenase enzyme as described above involved shuttling the product formed by the aldehyde dehydrogenase portion of the enzyme directly to the alcohol dehydrogenase portion of the enzyme. Prior to this discovery, and without wishing to be bound by theory, it was thought that a high-flux fermentation pathway that released a volatile or reactive intermediate, as could be the case if monofunctional enzymes were used, would both limit the yield of the pathway and prove toxic to the host cell.

However, Applicants have shown that host cells expressing a monofunctional aldehyde dehydrogenase and a monofunctional alcohol dehydrogenase are able to produce and accumulate alcohols such as butanol, for example, with the added benefit of limited production of ethanol, which is an undesirable side product. Further, the approach using monofunctional aldehyde dehydrogenases and monofunctional alcohol dehydrogenases allows for unique combinations of these monofunctional enzymes to be tailored for the production of particular alcohols.

In some embodiments, the methods and compositions as described herein involve a recombinant host cell that facilitates the production of an alcohol from an acyl-CoA, where the host cell includes: a first nucleic acid which encodes a polypeptide involved in the stepwise conversion of an acyl-CoA to a substrate for a monofunctional aldehyde dehydrogenase, a second nucleic acid which encodes a monofunctional aldehyde dehydrogenase, and a third nucleic acid which encodes a monofunctional alcohol dehydrogenase, where at least one of the first nucleic acid, the second nucleic acid, or the third nucleic acid is a recombinant nucleic acid. In some embodiments, at least two of the first nucleic acid, the second nucleic acid, and the third nucleic acid are separate nucleic acids (e.g. are located on separate plasmids in a host cell, or are separately encoded on the same plasmid).

Exemplary recombinant nucleic acids that encode a polypeptide involved in the stepwise conversion of an acyl-CoA to a substrate for a monofunctional aldehyde dehydrogenase which are suitable for use in the methods and compositions described herein include those which encode, for example, an acetoacetyl-CoA thiolase, a 3-hydroxybutyryl-CoA, a crotonase, and a trans-enoyl-CoA reductase. These polypeptides are described in more detail herein below.

Alcohols suitable for production from the recombinant host cells as described herein include, for example, C4 alcohols. Exemplary alcohols include saturated alcohols such as, for example n-butanol; unsaturated alcohols such as, for example, crotyl alcohol; diols such as, for example 1,3-butanediol; and the like. Typically, the alcohol is a C4 alcohol such as, for example, n-butanol, crotyl alcohol, 1,3-butanediol, and the like.

In other embodiments, recombinant host cells as described herein are capable of producing a C4 alcohol where the host cell contains a nucleic acid encoding an acetoacetyl-CoA thiolase, a nucleic acid encoding a 3-hydroxybutyryl-CoA dehydrogenase, a nucleic acid encoding a crotonase, a nucleic acid encoding a trans-enoyl-CoA reductase, a nucleic acid encoding a monofunctional aldehyde dehydrogenase, and a nucleic acid encoding a monofunctional alcohol dehydrogenase, where one or more of the nucleic acids is a recombinant nucleic acid. In some of these embodiments, at least two of the nucleic acids are recombinant or heterologous. In other embodiments, at least three, at least four, at least five, and in some instances all six of the nucleic acids are recombinant or heterologous. In some embodiments, the nucleic acid encoding the monofunctional aldehyde dehydrogenase and/or the nucleic acid encoding the monofunctional alcohol dehydrogenase is/are recombinant or heterologous.

In some embodiments, recombinant host cells as described herein are capable of producing n-butanol where the host cell contains a nucleic acid encoding an acetoacetyl-CoA thiolase capable of catalyzing the conversion of acetyl-CoA to acetoacetyl-CoA, a nucleic acid encoding a 3-hydroxybutyryl-CoA dehydrogenase capable of catalyzing the conversion of acetoacetyl-CoA to 3-hydroxybutyryl-CoA, a nucleic acid encoding a crotonase capable of catalyzing the conversion of 3-hydroxybutyryl-CoA to crotonyl-CoA, a nucleic acid encoding a trans-enoyl-CoA reductase capable of catalyzing the conversion of crotonyl-CoA to butyryl-CoA, a nucleic acid encoding a monofunctional aldehyde dehydrogenase capable of catalyzing the conversion of butyryl-CoA to butyraldehyde, and a nucleic acid encoding a monofunctional alcohol dehydrogenase capable of catalyzing the conversion of butyraldehyde to n-butanol, where one or more of the nucleic acids is a recombinant nucleic acid. In some of these embodiments, at least two of the nucleic acids are recombinant. In other embodiments, at least three, at least four, at least five, and in some instances all six of the nucleic acids are recombinant. In some embodiments, the nucleic acid encoding the monofunctional aldehyde dehydrogenase and/or the nucleic acid encoding the monofunctional alcohol dehydrogenase is/are recombinant or heterologous.

In some embodiments, recombinant host cells of the present disclosure are capable of producing crotyl alcohol where the host cell contains a nucleic acid encoding an acetoacetyl-CoA thiolase capable of catalyzing the conversion of acetyl-CoA to acetoacetyl-CoA, a nucleic acid encoding a 3-hydroxybutyryl-CoA dehydrogenase capable of catalyzing the conversion of acetoacetyl-CoA to 3-hydroxybutyryl-CoA, a nucleic acid encoding a crotonase capable of catalyzing the conversion of 3-hydroxybutyryl-CoA to crotonyl-CoA, a nucleic acid encoding a monofunctional aldehyde dehydrogenase capable of catalyzing the conversion of crotonyl-CoA to crotonaldehyde, and a nucleic acid encoding a monofunctional alcohol dehydrogenase capable of catalyzing the conversion of crotonaldehyde to crotyl alcohol, where one or more of the nucleic acids is a recombinant nucleic acid. In some of these embodiments, at least two of the nucleic acids are recombinant. In other embodiments, at least three, at least four, and in some instances all five of the nucleic acids are recombinant. In some embodiments, the nucleic acid encoding the monofunctional aldehyde dehydrogenase and/or the nucleic acid encoding the monofunctional alcohol dehydrogenase is/are recombinant or heterologous.

In some embodiments, recombinant host cells of the present disclosure are capable of producing 1,3-butanediol where the host cell contains a nucleic acid encoding an acetoacetyl-CoA thiolase capable of catalyzing the conversion of acetyl-CoA to acetoacetyl-CoA, a nucleic acid encoding a 3-hydroxybutyryl-CoA dehydrogenase capable of catalyzing the conversion of acetoacetyl-CoA to 3-hydroxybutyryl-CoA, a nucleic acid encoding a monofunctional aldehyde dehydrogenase capable of catalyzing the conversion of 3-hydroxybutyryl-CoA to 3-hydroxybutyraldehyde, and a nucleic acid encoding a monofunctional alcohol dehydrogenase capable of catalyzing the conversion of 3-hydroxybutyraldehyde to 1,3-butanediol, where one or more of the nucleic acids is recombinant or heterologous. In some of these embodiments, at least two of the nucleic acids are recombinant or heterologous. In other embodiments, at least three, and in some instances all four of the nucleic acids are recombinant or heterologous. In some embodiments, the nucleic acid encoding the monofunctional aldehyde dehydrogenase and/or the nucleic acid encoding the monofunctional alcohol dehydrogenase is/are recombinant or heterologous.

Recombinant host cells of the present disclosure may contain one or more nucleic acids encoding polypeptides such as, for example, any one of SEQ ID NOs: 1-154 and/or homologs thereof, and any one of SEQ ID NO: 250-266 and/or homologs thereof. Recombinant host cells of the present disclosure may contain one or more nucleic acids such as, for example, any one of SEQ ID NOs: 155-249, and/or homologs thereof.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### Polypeptides of the Present Disclosure

As described above, recombinant host cells of the present disclosure are engineered to contain one or more nucleic acids that encode polypeptides that are involved with and/or directly contribute to the biosynthesis of the alcohols described herein. These polypeptides, and the nucleic acids that encode them, are described in more detail below.

As used herein, a “polypeptide” is an amino acid sequence including a plurality of consecutive polymerized amino acid residues (e.g., at least about 15 consecutive polymerized amino acid residues). As used herein, “polypeptide” refers to an amino acid sequence, oligopeptide, peptide, protein, or portions thereof, and the terms “polypeptide” and “protein” are used interchangeably.

Proteins Involved in the Generation of Coenzyme a (CoA)

Alcohols of the present disclosure may be produced by a recombinant host cell via the stepwise conversion of the compound acetyl-CoA to an alcohol such as, for example, a C4 alcohol. As such, coenzyme A (CoA) may be used as a starting substrate in the production of acetyl-CoA. CoA may be endogenously present in the host cells or proteins involved in the generation of CoA may be recombinantly expressed in host cells such that the cells produce CoA. In host cells where CoA is endogenously present, various proteins may be modified and/or recombinantly expressed in the host cell such that the concentration of CoA in the host cell is modified (e.g. increased).

Various proteins are involved in the biosynthesis of coenzyme A. Proteins involved in the biosynthesis of CoA may be endogenously present or recombinantly expressed in a host cell of the present disclosure. Host cells of the present disclosure may contain, for example, a nucleic acid encoding a pantothenate kinase capable of catalyzing the conversion of pantothenate to 4′-phosphopantothenate. Pantothenate kinase may be derived from E. coli, or the pantothenate kinase may be PanK/CoaA or CoaX, for example. Host cells of the present disclosure may also contain, for example, a nucleic acid encoding a phosphopantothenoylcysteine synthetase capable of catalyzing the conversion of 4′-phosphopantothenate to 4′-phosphopantothenoylcysteine. Phosphopantothenoylcysteine synthetase may be derived from E. coli, or the phosphopantothenoylcysteine synthetase may be Ppcs or CoaB, for example. Host cells of the present disclosure may also contain, for example, a nucleic acid encoding a phosphopantothenonylcysteine decarboxylase capable of catalyzing the conversion of 4′-phosphopantothenoylcysteine to 4′-phosphopantetheine. Phosphopantothenonylcysteine decarboxylase may be derived from E. coli, or the phosphopantothenonylcysteine decarboxylase may be Ppcdc or CoaC, for example. Host cells of the present disclosure may also contain, for example, a nucleic acid encoding a phosphopantetheine adenylyl transferase capable of catalyzing the transfer of an adenylyl group from ATP to 4′-phosphopantetheine. Phosphopantetheine adenylyl transferase may be derived from E. coli, or the phosphopantetheine adenylyl transferase may be Ppat or CoaD, for example. Host cells of the present disclosure may also contain, for example, a nucleic acid encoding a dephosphocoenzyme A kinase capable of catalyzing the phosphorylation of dephospho-CoA. Dephosphocoenzyme A kinase may be derived from E. coli, or the dephosphocoenzyme A kinase may be CoaE, for example.

Recombinant nucleic acids encoding pantothenate kinase, phosphopantothenoylcysteine synthetase, phosphopantothenonylcysteine decarboxylase, phosphopantetheine adenylyl transferase, or dephosphocoenzyme A kinase may be derived from various prokaryotic organisms including, for example, proteobacterial, archaebacterial, bacteroidal, enterobacterial, spirochetal organisms. These nucleic acids may also be derived from various eukaryotic organisms including, for example, mammalian, insect, fungal and yeast organisms. The nucleic acids may be codon optimized to reflect the typical codon usage of the host cell, as described in more detail below.

Proteins Involved in the Generation of Acetyl-CoA

As described above, CoA may be endogenously present in the host cells of the present disclosure, or proteins involved in the generation of CoA may be recombinantly expressed in host cells such that the cells produce CoA. To produce acetyl-CoA, recombinant cells of the present disclosure may contain nucleic acids that encode at least one recombinant pathway for the production of acetyl-CoA. Acetyl-CoA can be generated from the glycolysis product pyruvate by way of, for example, a pyruvate dehydrogenase complex (PDHc), a pyruvate formate oxidoreductase (PFOR), the combined activities of a pyruvate formate lyase and a formate dehydrogenase (PFL-FDH), or a pyruvate dehydrogenase bypass pathway (PDH bypass). PDH bypass pathways may include, for example, a pyruvate dehydrogenase (PDH) in combination with an acylating aldehyde dehydrogenase or a non-acylating aldehyde dehydrogenase and an acetyl-CoA synthetase.

Recombinant host cells containing a pathway for the production of acetyl-CoA may contain, for example, recombinant polynucleotides encoding a pyruvate dehydrogenase complex (PDHc). In some embodiments, a pyruvate dehydrogenase complex is overexpressed in a host cell. In some embodiments, the PDH is Pdh from E. coli.

Recombinant host cells containing a pathway for the production of acetyl-CoA may contain, for example, recombinant or heterologous polynucleotides encoding a pyruvate formate lyase (PFL) and a formate dehydrogenase (FDH). Recombinant host cells containing a pathway for the production of acetyl-CoA may contain, for example, a recombinant polynucleotide encoding a pyruvate formate oxidoreductase complex (PFOR). In some embodiments, PFOR includes a pyruvate:flavodoxin/ferredoxin-oxidoreductase, a flavodoxin-NADP reductase, a ferredoxin, and at least one flavodoxin. In some embodiments, the recombinant proteins that compose the PFOR complex include YdbK, Fpr, Fdx, and FldA, or FldB from E. coli.

Recombinant host cells containing a pathway for the production of acetyl-CoA may contain, for example, one or more recombinant or heterologous nucleic acids encoding a pyruvate dehydrogenase bypass (PDH bypass). In some embodiments, the PDH bypass includes recombinant nucleic acids encoding a pyruvate decarboxylase (PDC). In some embodiments, the PDH bypass includes recombinant nucleic acids encoding a non-acylating aldehyde dehydrogenase. In some embodiments, the PDH bypass includes recombinant nucleic acids encoding an acetyl-CoA synthetase (ACS). In some embodiments, the PDHc bypass includes recombinant nucleic acids encoding a PDC, a non-acylating aldehyde dehydrogenase, and an ACS. In some embodiments, the PDH bypass includes recombinant nucleic acids encoding an acetylating aldehyde dehydrogenase. In some embodiments, the PDH bypass includes recombinant nucleic acids encoding a PDC and an acylating aldehyde dehydrogenase. In some embodiments, the PDH bypass includes recombinant nucleic acids encoding a PDC from Z. mobitilis and an acylating aldehyde dehydrogenase from E. coli. In some embodiments, the PDHc bypass contains recombinant nucleic acids encoding Pdc from Z. mobitilis and EutEA from E. coli.

Recombinant nucleic acids encoding PDHc, PFOR, PFL, FDH, acylating aldehyde dehydrogenase and non-acylating aldehyde dehydrogenase enzymes may be derived from various prokaryotic organisms including, for example, proteobacterial, archaebacterial, bacteroidal, enterobacterial, spirochetal organisms, as well as from various eukaryotic organisms including, for example, mammalian, insect, fungal and yeast organisms. The nucleic acids may be codon optimized to reflect the typical codon usage of the host cell, as described in more detail below. Exemplary nucleic acids include, for example, E. coli Pdh, which is composed of the three genes aceE, aceF, and lpdA; the E. faecalis Pdh, which is composed of the four genes pdhA, pdhB, aceF, and lpdA; the E. coli PFOR genes ydbK, fpr, fdx, fldA, and fldB; the Z. mobilis pdc gene; and the E. coli acetylating aldehyde dehydrogenase gene eutE. In some embodiments, the aceE protein has NCBI GenInfo Identifier Number GI 445925965 (SEQ ID NO: 38). In some embodiments, the aceF protein has NCBI GenInfo Identifier Number GI 446886262 (SEQ ID NO: 39). In some embodiments, the lpd protein has NCBI GenInfo Identifier Number GI 485653524 (SEQ ID NO: 40).

Acetoacetyl-CoA Thiolase

Certain aspects of the present disclosure relate to a recombinant host cell that contains a nucleic acid that encodes an acetoacetyl-CoA thiolase polypeptide, where the host cell may be used in the production of an alcohol in host cells. Acetoacetyl-CoA thiolase polypeptides are generally understood to be enzymes having E.C. 2.3.1.16 activity and that can catalyze the following reversible reaction: acyl-CoA+acetyl-CoA=CoA+3-oxoacyl-CoA. The acetoacetyl-CoA thiolase-encoding nucleic acids employed in the methods and compositions described herein may encode any of a variety of acetoacetyl-CoA thiolase polypeptides that are known in the art. The acetoacetyl-CoA thiolase polypeptide may be endogenously present or encoded by a heterologous polynucleotide (e.g., recombinantly expressed) in a host cell of the present disclosure. Recombinant nucleic acids encoding an acetoacetyl-CoA thiolase polypeptide may be derived from various prokaryotic organisms including, for example, proteobacterial, archaebacterial, bacteroidal, enterobacterial, spirochetal organisms, and various eukaryotic organisms including, for example, mammalian, insect, fungal and yeast organisms. The nucleic acids may be codon optimized to reflect the typical codon usage of the host cell, as described in more detail below. Examples of acetoacetyl-CoA thiolase polypeptides encoded by these nucleic acids include Ralstonia eutrophus acetoacetyl-CoA thiolase/synthase phaA and related enzymes from cells that make polyhydroxyalkanoates, C. acetobutylicum acetoacetyl-CoA thiolase/synthase thl, and E. coli acetoacetyl-CoA thiolase/synthase atoB. In some embodiments, the phaA protein has NCBI GenInfo Identifier Number GI 498509665 (SEQ ID NO: 33).

3-Hydroxybutyryl-CoA Dehydrogenase

Certain aspects of the present disclosure relate to a recombinant host cell that contains a nucleic acid that encodes a 3-hydroxybutyryl-CoA dehydrogenase polypeptide, where the host cell may be used in the production of an alcohol in host cells. 3-hydroxybutyryl-CoA dehydrogenase polypeptides are generally understood to be enzymes having E.C. 1.1.1.157 activity and that can catalyze, for example, the following reversible reaction: 3-hydroxybutanoyl-CoA+NADP+=3-acetoacetyl-CoA+NADPH+H+. The 3-hydroxybutanoyl-CoA dehydrogenase-encoding nucleic acids employed in the methods and compositions described herein may encode any of a variety of 3-hydroxybutyryl-CoA dehydrogenase polypeptides that are known in the art. The 3-hydroxybutyryl-CoA dehydrogenase polypeptide may be endogenously present or encoded by a heterologous polynucleotide (e.g., recombinantly expressed) in a host cell of the present disclosure. Recombinant nucleic acids encoding the 3-hydroxybutyryl-CoA dehydrogenase polypeptide may be derived from various prokaryotic organisms including, for example, proteobacterial, archaebacterial, bacteroidal, enterobacterial, spirochetal organisms, and various eukaryotic organisms including, for example, mammalian, insect, fungal and yeast organisms. The nucleic acids may be codon optimized to reflect the typical codon usage of the host cell, as described in more detail below. Examples of 3-hydroxybutyryl-CoA dehydrogenase polypeptides encoded by these nucleic acids include the R. eutrophus 3-hydroxybutyryl-CoA dehydrogenase phaB, the C. acetobutylicum acetoacetyl-CoA reductase hbd, and the 3-hydroxybutyryl-CoA dehydrogenase from Aeromonas caviae, hbd. In some embodiments, the phaB protein has NCBI GenInfo Identifier Number GI 113867453 (SEQ ID NO: 34). In some embodiments, the hbd protein has NCBI GenInfo Identifier Number GI 499268602 (SEQ ID NO: 35).

Crotonase

Certain aspects of the present disclosure relate to a recombinant host cell that contains a nucleic acid that encodes a crotonase polypeptide, where the host cell may be used in the production of an alcohol in host cells. Crotonase polypeptides are generally understood to be enzymes having E.C. 4.2.1.17 activity and that can catalyze, for example, the following reversible reaction: 3-hydroxyacyl-CoA=trans-2(or 3)-enoyl-CoA+H₂O. The crotonase-encoding nucleic acids employed in the methods and compositions described herein may encode any of a variety of crotonase polypeptides that are known in the art. The crotonase polypeptide may be endogenously present or encoded by a heterologous polynucleotide (e.g., recombinantly expressed) in a host cell of the present disclosure. Recombinant nucleic acid sequences encoding the crotonase polypeptide may be derived from various prokaryotic organisms including, for example, proteobacterial, archaebacterial, bacteroidal, enterobacterial, spirochetal organisms, and various eukaryotic organisms including, for example, mammalian, insect, fungal and yeast organisms. The nucleic acids may be codon optimized to reflect the typical codon usage of the host cell, as described in more detail below. Examples of crotonase polypeptides encoded by these nucleic acids include the C. acetobutylicum crotonase crt, and the A. cavaie crotonase phaJ. In some embodiments, the crt protein has NCBI GenInfo Identifier Number GI 15895969 (SEQ ID NO: 36).

Trans-Enoyl-CoA Reductase

Certain aspects of the present disclosure relate to the use of a recombinant host cell that contains a nucleic acid that encodes a trans-enoyl-CoA reductase polypeptide, where the host cell may be used in the production of an alcohol in host cells. Trans-enoyl-CoA reductase polypeptides are generally understood to be enzymes having E. C. 1.3.1.38 activity and that can catalyze, for example, the following reversible reaction: acyl-CoA+NADP+=trans-2,3-dehydroacyl-CoA+NADPH+H+. The trans-enoyl-CoA reductase-encoding nucleic acids employed in the methods and compositions described herein may encode any of a variety of trans-enoyl-CoA reductase polypeptides that are known in the art. The trans-enoyl-CoA reductase polypeptide may be endogenously present or encoded by a heterologous polynucleotide (e.g., recombinantly expressed) in a host cell of the present disclosure. Recombinant nucleic acids encoding the trans-enoyl-CoA reductase polypeptide may be derived from various prokaryotic organisms including, for example, proteobacterial, archaebacterial, bacteroidal, enterobacterial, spirochetal organisms, and various eukaryotic organisms including, for example, mammalian, insect, fungal and yeast organisms. The nucleic acids may be codon optimized to reflect the typical codon usage of the host cell, as described in more detail below. Examples of trans-enoyl-CoA reductase polypeptides encoded by these nucleic acids include trans-enoyl-CoA reductase polypeptides from T. denticola, E. gracilis, Burkholderia mallei, Burkholderia pseudomallei, Burkholderia cepacia, Methylobacillus flagellatus, Xylella fastidiosa, Xanthomonas campestris, Xanthomonas cryzae, Pseudomonas putida, Pseudomonas entomophila, Marinomonas sp., Psychromonas ingrahmii, Vibrio alginolyticus, Vibrio parahaemolyticus, Vibrio splendidus, Vibrio sp., Shewanella frigidimarina, Oceanospirillum sp., Aeromonas hydrophila subsp., Serratiae proteamaculans, Saccharophagus degradans, Colwellia psychrerythraea, Reine kea sp., Idiomarina loihiensis, Streptomyces avermitilis, Coxiella burnetii Dugway, Polaribacter irgensii, Flavobacterium johnsoniae, Cytophaga hutchisonii, E. coli, R. eutrophus, A. caviae, and C. acetobutylicum. In some embodiments, the ter protein has NCBI GenInfo Identifier Number GI 488758537 (SEQ ID NO: 37).

Monofunctional Aldehyde Dehydrogenase

Certain aspects of the present disclosure relate to a recombinant host cell that contains a nucleic acid that encodes a monofunctional aldehyde dehydrogenase polypeptide for use in the production of an alcohol in host cells. Aldehyde dehydrogenase polypeptides are generally understood to be enzymes having E. C. 1.2.1.10 activity. Aldehyde dehydrogenase polypeptides of the present disclosure may be used to catalyze the conversion of a CoA-containing molecule to an aldehyde, using NADH or NADPH as a cofactor. A monofunctional aldehyde dehydrogenase polypeptide of the present disclosure may be used to catalyze the conversion of butyryl-CoA to butyraldehyde. A monofunctional aldehyde dehydrogenase polypeptide of the present disclosure may be used to catalyze the conversion of crotonyl-CoA to crotonaldehyde. A monofunctional aldehyde dehydrogenase polypeptide of the present disclosure may be used to catalyze the conversion of 3-hydroxybutyryl-CoA to 3-hydroxybutyraldehyde. In certain embodiments, a host cell comprises a heterologous polynucleotide encoding a polypeptide having monofunctional aldehyde dehydrogenase activity.

The monofunctional aldehyde dehydrogenase-encoding nucleic acid may encode any of a variety of aldehyde dehydrogenases that are known in the art. The nucleic acid may be codon optimized to reflect the typical codon usage of the host cell, as described in more detail below. The encoded aldehyde dehydrogenase may be, for example, an aldehyde dehydrogenase having NCBI GenInfo Identifier Number GI 4884855 (SEQ ID NO: 1), GI 26250354 (SEQ ID NO: 2), GI 31075383 (SEQ ID NO: 3), GI 149190407 (SEQ ID NO: 4), GI 154503198 (SEQ ID NO: 5), GI 160942363 (SEQ ID NO: 6), GI 187934965 (SEQ ID NO: 7), GI 189310620 (SEQ ID NO: 8), GI 251780016 (SEQ ID NO: 9), GI 255526882 (SEQ ID NO: 10), GI 302386203 (SEQ ID NO: 11), GI 312110932 (SEQ ID NO: 12), GI 359413662 (SEQ ID NO: 13), GI 371960349 (SEQ ID NO: 14), GI 373496187 (SEQ ID NO: 15), and GI 150018649 (SEQ ID NO: 16).

Monofunctional Alcohol Dehydrogenase

Certain aspects of the present disclosure relate to a recombinant host cell that contains a nucleic acid that encodes a monofunctional alcohol dehydrogenase polypeptide, where the host cell may be used in the production of an alcohol. Alcohol dehydrogenase polypeptides are generally understood to be enzymes having E. C. 1.1.1.1 activity. Alcohol dehydrogenase polypeptides of the present disclosure may be used to catalyze the conversion of an aldehyde into an alcohol, using NADH or NADPH as a cofactor. A monofunctional alcohol dehydrogenase polypeptide of the present disclosure may be used to catalyze the conversion of butyraldehyde to n-butanol. A monofunctional alcohol dehydrogenase polypeptide of the present disclosure may be used to catalyze the conversion of crotonaldehyde to crotyl alcohol. A monofunctional alcohol dehydrogenase polypeptide of the present disclosure may be used to catalyze the conversion of 3-hydroxybutyraldehyde to 1,3-butanediol. In certain embodiments, a host cell comprises a heterologous polynucleotide encoding a polypeptide having monofunctional alcohol dehydrogenase activity.

The monofunctional alcohol dehydrogenase-encoding nucleic acid may encode any of a variety of alcohol dehydrogenases that are known in the art. The nucleic acid may be codon optimized to reflect the typical codon usage of the host cell, as described in more detail below. The encoded alcohol dehydrogenase may be, for example, an alcohol dehydrogenase having UniProt ID A0RQF7_CAMFF (SEQ ID NO: 17), G5F136_9ACTN (SEQ ID NO: 18), B1C7G7_9FIRM (SEQ ID NO: 19), YUGK_BACSU (SEQ ID NO: 20), A8SGI9_9FIRM (SEQ ID NO: 21), E2SQ66_9FIRM (SEQ ID NO: 22), E1QYZ8_OLSUV (SEQ ID NO: 23), F5X0G1_STRG1 (SEQ ID NO: 24), E6W4G5_DESIS (SEQ ID NO: 25), B1C4Z8_9FIRM (SEQ ID NO: 26), G4L3E3_TETHN (SEQ ID NO: 27), E8LLW8_9GAMM (SEQ ID NO: 28), E4RKV2_HALHG (SEQ ID NO: 29), Q15G22_CITFR (SEQ ID NO: 30), AOPY50_CLONN (SEQ ID NO: 31), and Q3A1K9_PELCD (SEQ ID NO: 32).

Certain aspects of the present disclosure also relate to a recombinant host cell that contains a nucleic acid that encodes a monofunctional secondary alcohol dehydrogenase polypeptide, where the host cell may be used in the production of an alcohol. Secondary alcohol dehydrogenase polypeptides are generally understood to be enzymes having E. C. 1.1.1.1 activity. Secondary alcohol dehydrogenases of the present disclosure may be used e.g. in the conversion of 4-hydroxy-2-butanone to 1,3-butanediol. In certain embodiments, a host cell comprises a heterologous polynucleotide encoding a polypeptide having monofunctional secondary alcohol dehydrogenase activity.

The monofunctional secondary alcohol dehydrogenase-encoding nucleic acid may encode any of a variety of secondary alcohol dehydrogenases that are known in the art. The nucleic acid may be codon optimized to reflect the typical codon usage of the host cell, as described in more detail below. The encoded secondary alcohol dehydrogenase may be, for example, a secondary alcohol dehydrogenase selected from Table 5 herein, including SEQ ID NOs: 250-266.

AdhE2 Variants

Certain aspects of the present disclosure relate to a recombinant host cell that contains a nucleic acid that encodes a variant of a wild-type AdhE2 polypeptide. Accordingly, further provided herein are variants of a wild-type AdhE2 polypeptide (SEQ ID NO: 41). Variants of a wild-type AdhE2 polypeptide may include, for example, a polypeptide having the amino acid sequence of any one of SEQ ID NOs: 60-154. Nucleic acids encoding variants of a wild-type AdhE2 polypeptide may include, for example, any one of SEQ ID NO: 155-249.

Sequence Similarity to Polypeptides of the Disclosure

Nucleic acids suitable for use in the methods and compositions described herein include those that encode, for example, any known or putative protein involved in the biosynthesis of coenzyme A (CoA), any known or putative protein involved in the biosynthesis of acetyl-CoA, and any known or putative acetoacetyl-CoA thiolase, 3-hydroxybutyryl-CoA dehydrogenase, crotonase, trans-enoyl-CoA reductase, monofunctional aldehyde dehydrogenase, and/or monofunctional alcohol dehydrogenase, also include polypeptides that are homologs and/or orthologs of the polypeptides described herein. Methods for the identification of polypeptides that are homologs of a polypeptide of interest are well-known to one of skill in the art, as described herein.

In some embodiments, the encoded polypeptides have an amino acid sequence that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to the amino acid sequence of any known or putative polypeptide described herein such as, for example, any known or putative protein involved in the biosynthesis of coenzyme A (CoA), any known or putative protein involved in the biosynthesis of acetyl-CoA, and any known or putative acetoacetyl-CoA thiolase, 3-hydroxybutyryl-CoA dehydrogenase, crotonase, trans-enoyl-CoA reductase, monofunctional aldehyde dehydrogenase, and/or monofunctional alcohol dehydrogenase.

The polypeptides used in the methods and compositions described herein may include, for example, a polypeptide having an amino acid sequence that has at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, and/or SEQ ID NO: 266.

In some embodiments, the encoded polypeptides have at least 10, at least 12, at least 14, at least 16, at least 18, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, or at least 250 consecutive amino acids of any known or putative polypeptide described herein such as, for example, any known or putative protein involved in the biosynthesis of coenzyme A (CoA), any known or putative protein involved in the biosynthesis of acetyl-CoA, and any known or putative acetoacetyl-CoA thiolase, 3-hydroxybutyryl-CoA dehydrogenase, crotonase, trans-enoyl-CoA reductase, monofunctional aldehyde dehydrogenase, and/or monofunctional alcohol dehydrogenase.

The polypeptides used in the methods and compositions described herein may include, for example, a polypeptide having least 10, at least 12, at least 14, at least 16, at least 18, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, or at least 250 consecutive amino acids of the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, and/or SEQ ID NO: 266.

The encoded polypeptides that include, for example, any known or putative protein involved in the biosynthesis of coenzyme A (CoA), any known or putative protein involved in the biosynthesis of acetyl-CoA, and any known or putative acetoacetyl-CoA thiolase, 3-hydroxybutyryl-CoA dehydrogenase, crotonase, trans-enoyl-CoA reductase, monofunctional aldehyde dehydrogenase, and/or monofunctional alcohol dehydrogenase, also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

### Polynucleotides Encoding Polypeptides

As described above, the present disclosure further relates to polynucleotides that encode polypeptides present in the host cells as described herein. For example, polynucleotides encoding any known or putative protein involved in the biosynthesis of coenzyme A (CoA), any known or putative protein involved in the biosynthesis of acetyl-CoA, and any known or putative acetoacetyl-CoA thiolase, 3-hydroxybutyryl-CoA dehydrogenase, crotonase, trans-enoyl-CoA reductase, monofunctional aldehyde dehydrogenase, and/or monofunctional alcohol dehydrogenase as described herein are provided. Methods for determining the relationship between a polypeptide and a polynucleotide that encodes the polypeptide are well-known to one of skill in the art. Similarly, methods of determining the polypeptide sequence encoded by a polynucleotide sequence are well-known to one of skill in the art.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

The nucleic acids employed in the methods and compositions described herein may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, where each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those of ordinary skill in the art, such as utilization of polymerase chain reactions (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g. a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g. bacterial cell, mammalian cell, etc.). Methods of codon optimizing a nucleic acid for tailored expression in a particular cell type are well-known to those of skill in the art.

A polynucleotide encoding a polypeptide used in the methods and compositions described herein may include, for example, a polynucleotide that encodes a polypeptide having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, and/or SEQ ID NO: 266.

A polynucleotide encoding an AdhE2 variant of the present disclosure may include, for example, a polynucleotide having at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% identity to the nucleotide sequence of SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, and/or SEQ ID NO: 249.

### Methods of Identifying Sequence Similarity

As described above, various polynucleotides and/or polypeptides that are similar to the polynucleotides and/or polypeptides as described herein may be used in the compositions and methods as described herein. Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e. by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g. charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer assisted methods. As noted below, a variety of computer programs for performing sequence alignment are available, or can be produced by one of skill.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul, Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif.); the AlignX program, version10.3.0 (Invitrogen, Carlsbad, Calif.) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. J. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Hames and Higgins, ed., Nucleic Acid Hybridisation, A Practical Approach. Oxford, TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full length cDNA, homologs, orthologs, and paralogs of polynucleotides of the present disclosure may be identified and isolated using well-known polynucleotide hybridization methods.

### Vectors for Expressing Polynucleotides

The recombinant polynucleotides employed in the methods and compositions described herein may be incorporated into an expression vector. “Expression vector” or “vector” refers to a compound and/or composition that transduces, transforms, or infects a host cell, thereby causing the cell to express polynucleotides and/or proteins other than those native to the cell, or in a manner not native to the cell. An “expression vector” contains a sequence of polynucleotides (ordinarily RNA or DNA) to be expressed by the host cell. Optionally, the expression vector also includes materials to aid in achieving entry of the polynucleotide into the host cell, such as a virus, liposome, protein coating, or the like. The expression vectors contemplated for use in the present disclosure include those into which a polynucleotide sequence can be inserted, along with any preferred or required operational elements. Further, the expression vector must be one that can be transferred into a host cell and replicated therein. Preferred expression vectors are plasmids, particularly those with restriction sites that have been well-documented and that contain the operational elements preferred or required for transcription of the polynucleotide sequence. Such plasmids, as well as other expression vectors, are well-known in the art.

Incorporation of the individual polynucleotides may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a polynucleotide having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide are often cleaved with the same restriction enzyme, thereby assuring that the ends of the expression vector and the ends of the polynucleotide are complementary to each other. In addition, DNA linkers may be used to facilitate linking of polynucleotide sequences into an expression vector.

A series of individual polynucleotides can also be combined by utilizing methods that are known in the art (e.g., U.S. Pat. No. 4,683,195). For example, each of the desired polynucleotides can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual polynucleotides may be “spliced” together and subsequently transduced into a host cell simultaneously. Thus, expression of each of the plurality of polynucleotides is affected.

Individual polynucleotides, or “spliced” polynucleotides, are then incorporated into an expression vector. The present disclosure is not limited with respect to the process by which the polynucleotide is incorporated into the expression vector. Those of ordinary skill in the art are familiar with the necessary steps for incorporating a polynucleotide into an expression vector. A typical expression vector contains the desired polynucleotide preceded by one or more regulatory regions, along with a ribosome binding site, e.g., a nucleotide sequence that is 3-9 nucleotides in length and located 3-11 nucleotides upstream of the initiation codon in E. coli. See Shine and Dalgarno (1975) Nature 254(5495):34-38 and Steitz (1979) Biological Regulation and Development (ed. Goldberger, R. F.), 1:349-399 (Plenum, N.Y.).

The term “operably linked” as used herein refers to a configuration in which a control sequence is placed at an appropriate position relative to the coding sequence of the DNA sequence or polynucleotide such that the control sequence directs the expression of a polypeptide.

Regulatory regions include, for example, those regions that contain a promoter and an operator. A promoter is operably linked to the desired polynucleotide, thereby initiating transcription of the polynucleotide via an RNA polymerase enzyme. An operator is a sequence of polynucleotides adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see de Boer et al., (1983) Proc Natl Acad Sci USA 80(1):21-25).

Methods of producing host cells of the disclosure may include the introduction or transfer of the expression vectors containing recombinant nucleic acids of the disclosure into the host cell. Such methods for transferring expression vectors into host cells are well-known to those of ordinary skill in the art. For example, one method for transforming cells with an expression vector involves a calcium chloride treatment where the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Cells also may be transformed through the use of spheroplasts (Schweizer, M, Proc. Natl. Acad. Sci., 78: 5086-5090 (1981). Also, microinjection of the nucleic acid sequences provides the ability to transfect host cells. Other means, such as lipid complexes, liposomes, and dendrimers, may also be employed. Those of ordinary skill in the art can transfect a host cell with a desired sequence using these or other methods.

In some cases, cells are prepared as protoplasts or spheroplasts prior to transformation. Protoplasts or spheroplasts may be prepared, for example, by treating a cell having a cell wall with enzymes to degrade the cell wall. Fungal cells may be treated, for example, with chitinase.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated. Furthermore, a single vector or plasmid or two or more vectors or plasmids that together contain the total DNA to be introduced into the genome of the host, or a transposon may be used.

The vectors preferably contain one or more selectable markers which permit easy selection of transformed host cells. A selectable marker is a gene the product of which provides, for example, biocide or viral resistance, resistance to heavy metals, prototrophy to auxotrophs, and the like. Selection of bacterial cells may be based upon antimicrobial resistance that has been conferred by genes such as the amp, gpt, neo, and hyg genes.

Selectable markers for use in fungal host cells may include, for example, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof.

The vectors may contain an element(s) that permits integration of the vector into the host's genome or autonomous replication of the vector in the cell independent of the genome.

For integration into the host genome, the vector may rely on the gene's sequence or any other element of the vector for integration of the vector into the genome by homologous or nonhomologous recombination. Alternatively, the vector may contain additional nucleotide sequences for directing integration by homologous recombination into the genome of the host. The additional nucleotide sequences enable the vector to be integrated into the host genome at a precise location(s) in the chromosome(s). To increase the likelihood of integration at a precise location, the integrational elements should contain a sufficient number of nucleic acids, such as 100 to 10,000 base pairs, 400 to 10,000 base pairs, or 800 to 10,000 base pairs, which are highly homologous with the corresponding target sequence to enhance the probability of homologous recombination. The integrational elements may be any sequence that is homologous with the target sequence in the genome of the host. Furthermore, the integrational elements may be non-encoding or encoding nucleotide sequences. On the other hand, the vector may be integrated into the genome of the host by non-homologous recombination.

For autonomous replication, the vector may further contain an origin of replication enabling the vector to replicate autonomously in the host in question. The origin of replication may be any plasmid replicator mediating autonomous replication which functions in a cell. The term “origin of replication” or “plasmid replicator” is defined herein as a sequence that enables a plasmid or vector to replicate in vivo.

Various promoters for regulation of expression of a recombinant nucleic acid of the disclosure in a vector are well-known in the art and include, for example, constitutive promoters and inducible promoters. Promoters are described, for example, in Sambrook, et al. Molecular Cloning: A Laboratory Manual, 3rd edition, Cold Spring Harbor Laboratory Press, (2001). Promoter can be viral, bacterial, fungal, mammalian, or plant promoters. Additionally, promoters can be constitutive promoters, inducible promoters, environmentally regulated promoters, or developmentally regulated promoters. Examples of suitable promoters for regulating recombinant nucleic acid of the disclosure may include, for example, the N. crassa ccg-1 constitutive promoter, which is responsive to the N. crassa circadian rhythm and nutrient conditions; the N. crassa gpd-1 (glyceraldehyde 3-phosphate dehydrogenase-1) strong constitutive promoter; the N. crassa vvd (light) inducible promoter; the N. crassa qa-2 (quinic acid) inducible promoter; the Aspergillus nidulans gpdA promoter; the Aspergillus nidulans trpC constitutive promoter; the N. crassa tef-1 (transcription elongation factor) highly constitutive promoter; and the N. crassa xlr-1 (XlnR homolog) promoter, which is used frequently in Aspergillus species. In some embodiments, expression of a recombinant polypeptide of the disclosure is under the control of a heterologous promoter.

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

The procedures used to ligate the elements described above to construct the recombinant expression vectors of the present disclosure are well-known to one skilled in the art (see, e.g., Sambrook et al., 1989, supra). When only a single expression vector is used (without the addition of an intermediate), the vector will contain all of the nucleic acid sequences necessary.

### Host Cells of the Disclosure

Host cells of the present disclosure may include various prokaryotic cells such as, for example, proteobacterial, archaebacterial, bacteroidal, enterobacterial, and spirochetal cells, as well as various eurkaryotic cells such as, for example, mammalian, insect, fungal and yeast cell types. Host cells of the present disclosure may be, for example, E. coli cells, Zymomonas mobilis (Z. mobilis) cells, Bacillus subtilis (B. subtilis) cells, yeast cells including S. cerevisiae cells and S. pombe cells, cyanobacterial cells such as Synechocystis sp. and Synechococcus sp., photosynthetic cells such as Rhodospirillum sp., solvent producing cells such as Clostridium sp. (such as, for example, Clostridium acetobutylicum and Clostridium beijerinckii), chemoautotrophic cells such as Ralstonia sp., in general and Ralstonia eutrophus for example, aromatic-degrading cells such as Pseudomonas sp. and Rhodococcus sp., thermophilic cells such as Thermoanaerobacterium saccharolyticum (T. saccharolyticum) and Thermotoga sp., cellulolytic cells such as Trichoderma reesei (T. reesei) cells, and Aspergillus niger (A. niger) cells, and lignocellulolytic cells such as Phanerochaete chrysosporium (P. chrysosporium).

Host cells of the present disclosure are living biological cells that may be manipulated to exhibit characteristics that differ from a corresponding control cell such as, for example, a naturally occurring wild-type cell. For example, host cells may be transformed via insertion of recombinant or heterologous DNA or RNA. Such recombinant DNA or RNA can be in an expression vector. Further, host cells may be subjected to mutagenesis to induce mutations in polypeptide-encoding polynucleotides. Host cells that have been genetically modified or engineered are recombinant host cells.

The host cells of the present disclosure may be genetically modified or engineered. For example, recombinant or heterologous nucleic acids may have been introduced into the host cells or the host cells may have mutations introduced into endogenous and/or exogenous polynucleotides, and as such the genetically modified or engineered host cells do not occur in nature. A suitable host cell may be, for example, one that is capable of expressing one or more nucleic acid constructs for different functions such as, for example, recombinant protein expression and/or targeted gene silencing.

“Recombinant nucleic acid” or “heterologous nucleic acid” or “recombinant polynucleotide”, “recombinant nucleotide” or “recombinant DNA” as used herein refers to a polymer of nucleic acids where at least one of the following is true: (a) the nucleic acid molecule is foreign to (i.e., not naturally found in) a given host cell; (b) the nucleic acid molecule may be naturally found in a given host cell, but its product is expressed in an unnatural (e.g., greater than expected) amount; or (c) the nucleic acid molecule contains two or more subsequences that are not found in the same relationship to each other in nature, wherein such alterations or modifications are introduced by genetic engineering. For example, regarding instance (c), a recombinant nucleic acid sequence will have two or more sequences from unrelated genes arranged to make a new functional nucleic acid. For example, the present disclosure describes the introduction of an expression vector into a host cell, where the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant. As used herein, the term “recombinant polypeptide” refers to a polypeptide generated from a “recombinant nucleic acid” or “heterologous nucleic acid” or “recombinant polynucleotide”, “recombinant nucleotide” or “recombinant DNA” as described above.

In some embodiments, the host cell naturally produces one or more of the polypeptides of the present disclosure. In some embodiments, the genes encoding the desired polypeptides may be heterologous to the host cell or these genes may be endogenous to the host cell but are operatively linked to heterologous promoters and/or control regions that result in, for example, the higher expression of the gene(s) in the host cell or the decreased expression of the gene(s) in the host cell.

Host cells of the present disclosure may contain enzymes or other proteins having reduced activity as compared to a corresponding control cell, where those proteins may directly or indirectly negatively impact the production of alcohols by the host cell. For examples, proteins that may have reduced activity as compared to a corresponding control cell may be certain enzymes in pathways that utilize pyruvate or acetyl-CoA to synthesize products other than an alcohol.

One of skill in the art would readily recognize an appropriate corresponding control cell for use in a given comparison to a host cell of the present disclosure. For example, a corresponding control cell may be a wild-type cell. A corresponding control cell could include, for example, a parental cell, such that the parental cell is being compared to a child cell where some genetic modification has been made relative to the parental cell. A corresponding control cell could also include, for example, a cell similar to a host cell of the present disclosure that contains a bifunctional aldehyde/alcohol dehydrogenase, as opposed to a separate monofunctional aldehyde dehydrogenase and a monofunctional alcohol dehydrogenase.

In some embodiments, host cells of the present disclosure have reduced or eliminated activity of protein activities involved in the synthesis of lactate from pyruvate as compared to a corresponding control cell. In some embodiments, host cells of the present disclosure have reduced or eliminated activity of protein activities involved in the synthesis of acetate from acetyl-CoA as compared to a corresponding control cell. In some embodiments, host cells of the present disclosure have reduced or eliminated activity of protein activities involved in the synthesis of ethanol from acetyl-CoA as compared to a corresponding control cell.

In some embodiment, the host cell contains a lactate dehydrogenase that catalyzes the conversion of pyruvate to lactate with reduced or eliminated activity as compared to a corresponding control cell. The lactate dehydrogenase may be, for example, ldhA from E. coli. In some embodiments, the host cell contains a pyruvate oxidase that catalyzes the conversion of pyruvate to acetate with reduced or eliminated activity as compared to a corresponding control cell. The pyruvate oxidase may be, for example, poxB from E. coli. In some embodiments, the host cell contains an alcohol dehydrogenase that catalyzes the conversion of acetyl-CoA to ethanol with reduced or eliminated activity as compared to a corresponding control cell. The alcohol dehydrogenase may be, for example, adhE from E. coli. In some embodiments, the host cell contains an acetate kinase that catalyzes the conversion of acetyl-CoA to acetate with reduced or eliminated activity as compared to a corresponding control cell. The acetate kinase may be, for example, ackA. In some embodiments, the host cell contains a phosphotransacetylase that catalyzes the conversion of acetyl-CoA to acetate with reduced or eliminated activity as compared to a corresponding control cell. The phosphotransacetylase may be, for example, pta. In some embodiments, the host cell contains a fumarate dehydrogenase that catalyzes the conversion of succinate to fumarate with reduced or eliminated activity as compared to a corresponding control cell. The fumarate dehydrogenase may be, for example, frd from E. coli.

The activity of a protein or enzyme having reduced or eliminated activity may be reduced by at least about 5%, 10%, 20%, 30%, 40%, 50%, 60%, 70%, 80%, 90%, or 100% as compared to a corresponding control cell. Mutations reducing or eliminating the activity of proteins or enzymes may include, for example, point mutations that cause amino acid changes in the enzymes, deletion mutations, nonsense mutations, frameshift mutations, sequence duplications or inversions and insertions. Mutations may be introduced in a targeted or non-targeted manner. Reducing the activity of a protein or enzyme may also be introduced, either directly or indirectly, by molecular biology means such as, for example, the use of homologous recombinations, antisense technologies or RNA interference, or by chemical means, such as treatments with DNA intercalators or DNA methylating agents.

Methods of decreasing the expression, abundance, and/or activity of a polypeptide are well-known in the art and are described herein.

In some embodiments, decreasing activity of a polypeptide involves overexpressing a polypeptide that is an inhibitor of the polypeptide. Host cells may overexpress an inhibitor that inhibits the expression and/or activity of a polypeptide of the present disclosure. In some embodiments, a recombinant polypeptide may be expressed in host cells such that the recombinant polypeptide interferes with and decreases the activity of the endogenous polypeptide. In some embodiments, decreasing the activity of a polypeptide involves decreasing the expression of a nucleic acid encoding the polypeptide.

Mutagenesis approaches may be used to disrupt or “knockout” the expression of a target gene by generating mutations. In some embodiments, the mutagenesis results in a partial deletion of the target gene. In other embodiments, the mutagenesis results in a complete deletion of the target gene. Methods of mutagenizing microorganisms are well known in the art and include, for example, random mutagenesis and site-directed mutagenesis to induce mutations. Examples of methods of random mutagenesis include, for example, chemical mutagenesis (e.g., using ethane methyl sulfonate), insertional mutagenesis, and irradiation.

One method for reducing or inhibiting the expression of a target gene is by genetically modifying or engineering the target gene and introducing it into the genome of a host cell to replace the wild-type version of the gene by homologous recombination (for example, as described in U.S. Pat. No. 6,924,146).

Another method for reducing or inhibiting the expression of a target gene is by insertion mutagenesis using the T-DNA of Agrobacterium tumefaciens, or transposons (see Winkler et al., Methods Mol. Biol. 82:129-136, 1989, and Martienssen Proc. Natl. Acad. Sci. 95:2021-2026, 1998). After generating the insertion mutants, the mutants can be screened to identify those containing the insertion in a target gene. Methods to disrupt a target gene by insertional mutagenesis are described in for example, U.S. Pat. No. 5,792,633. Methods to disrupt a target gene by transposon mutagenesis are described in for example, U.S. Pat. No. 6,207,384.

A further method to disrupt a target gene is by use of the cre-lox system (for example, as described in U.S. Pat. No. 4,959,317). Another method to disrupt a target gene is by use of PCR mutagenesis (for example, as described in U.S. Pat. No. 7,501,275). Endogenous gene expression may also be reduced or inhibited by means of RNA interference (RNAi), which uses a double-stranded RNA having a sequence identical or similar to the sequence of the target gene. RNAi may include the use of micro RNA, such as artificial miRNA, to suppress expression of a gene.

RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA.

Thus, in some embodiments, reduction or inhibition of gene expression is achieved using RNAi techniques. For example, to achieve reduction or inhibition of the expression of a DNA encoding a protein using RNAi, a double-stranded RNA having the sequence of a DNA encoding the protein, or a substantially similar sequence thereof (including those engineered not to translate the protein) or fragment thereof, is introduced into a host cell of interest. As used herein, RNAi and dsRNA both refer to gene-specific silencing that is induced by the introduction of a double-stranded RNA molecule, see e.g., U.S. Pat. Nos. 6,506,559 and 6,573,099, and includes reference to a molecule that has a region that is double-stranded, e.g., a short hairpin RNA molecule. The resulting cells may then be screened for a phenotype associated with the reduced expression of the target gene, e.g., reduced cellulase expression, and/or by monitoring steady-state RNA levels for transcripts of the target gene. Although the sequences used for RNAi need not be completely identical to the target gene, they may be at least 70%, 80%, 90%, 91%, 92%, 93%, 94%, 95%, 96%, 97%, 98%, 99%, or more identical to the target gene sequence. See, e.g., U.S. Patent Application Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Application Publication No. 2003/0221211.

The RNAi nucleic acids may encompass the full-length target RNA or may correspond to a fragment of the target RNA. In some cases, the fragment will have fewer than 100, 200, 300, 400, or 500 nucleotides corresponding to the target sequence. In addition, in some aspects, these fragments are at least, e.g., 50, 100, 150, 200, or more nucleotides in length. Interfering RNAs may be designed based on short duplexes (i.e., short regions of double-stranded sequences). Typically, the short duplex is at least about 15, 20, or 25-50 nucleotides in length (e.g., each complementary sequence of the double stranded RNA is 15-50 nucleotides in length), often about 20-30 nucleotides, e.g., 20, 21, 22, 23, 24, 25, 26, 27, 28, 29, or 30 nucleotides in length. In some cases, fragments for use in RNAi will correspond to regions of a target protein that do not occur in other proteins in the organism or that have little similarity to other transcripts in the organism, e.g., selected by comparison to sequences in analyzing publicly-available sequence databases. Similarly, RNAi fragments may be selected for similarity or identity with a conserved sequence of a gene family of interest, such as those described herein, so that the RNAi targets multiple different gene transcripts containing the conserved sequence.

RNAi may be introduced into a host cell as part of a larger DNA construct. Often, such constructs allow stable expression of the RNAi in cells after introduction, e.g., by integration of the construct into the host genome. Thus, expression vectors that continually express RNAi in cells transfected with the vectors may be employed for this disclosure. For example, vectors that express small hairpin or stem-loop structure RNAs, or precursors to microRNA, which get processed in vivo into small RNAi molecules capable of carrying out gene-specific silencing (Brummelkamp et al, Science 296:550-553, (2002); and Paddison, et al., Genes & Dev. 16:948-958, (2002)) can be used. Post-transcriptional gene silencing by double-stranded RNA is discussed in further detail by Hammond et al., Nature Rev Gen 2: 110-119, (2001); Fire et al., Nature 391: 806-811, (1998); and Timmons and Fire, Nature 395: 854, (1998). Methods for selection and design of sequences that generate RNAi are well-known in the art (e.g. U.S. Pat. Nos. 6,506,559; 6,511,824; and 6,489,127).

A reduction or inhibition of gene expression in a host cell of a target gene may also be obtained by introducing into host cells antisense constructs based on a target gene nucleic acid sequence. For antisense suppression, a target sequence is arranged in reverse orientation relative to the promoter sequence in the expression vector. The introduced sequence need not be a full length cDNA or gene, and need not be identical to the target cDNA or a gene found in the cell to be transformed. Generally, however, where the introduced sequence is of shorter length, a higher degree of homology to the native target sequence is used to achieve effective antisense suppression. In some aspects, the introduced antisense sequence in the vector will be at least 30 nucleotides in length, and improved antisense suppression will typically be observed as the length of the antisense sequence increases. In some aspects, the length of the antisense sequence in the vector will be greater than 100 nucleotides. Transcription of an antisense construct as described results in the production of RNA molecules that are the reverse complement of mRNA molecules transcribed from an endogenous target gene. Suppression of a target gene expression can also be achieved using a ribozyme. The production and use of ribozymes are disclosed in U.S. Pat. Nos. 4,987,071 and 5,543,508.

Expression cassettes containing nucleic acids that encode target gene expression inhibitors, e.g., an antisense or siRNA, can be constructed using methods well known in the art. Constructs include regulatory elements, including promoters and other sequences for expression and selection of cells that express the construct. Typically, fungal and/or bacterial transformation vectors include one or more cloned coding sequences (genomic or cDNA) under the transcriptional control of 5′ and 3′ regulatory sequences and a dominant selectable marker. Such transformation vectors typically also contain a promoter (e.g., a regulatory region controlling inducible or constitutive, environmentally- or developmentally-regulated expression), a transcription initiation start site, an RNA processing signal (such as intron splice sites), a transcription termination site, and/or a polyadenylation signal.

Host cells of the present disclosure may contain one or more polypeptides with increased activity as compared to a corresponding control cell. Various methods of increasing polypeptide activity are well-known in the art and are described herein. In certain embodiments, a recombinant nucleic acid is mis-expressed in the host cell (e.g., constitutively expressed, inducibly expressed, etc.) such that mis-expression results in increased polypeptide activity as compared to a corresponding control cell. In some embodiments, a host cell that contains a recombinant nucleic acid encoding a recombinant polypeptide contains a greater amount of the polypeptide than a corresponding control cell that does not contain the corresponding recombinant nucleic acid. When a protein or nucleic acid is produced or maintained in a host cell at an amount greater than normal, the protein or nucleic acid is “overexpressed.” The corresponding control cell may be, for example, a cell that does not overexpress one or more of the polypeptides overexpressed in the host cell. Various control cells will be readily apparent to one of skill in the art, as described above.

Various methods of increasing the expression of a polypeptide are known in the art. For example, other genetic regions involved in controlling expression of the nucleic acid encoding the polypeptide, such as an enhancer sequence, may be modified such that expression of the nucleic acid is increased. The level of expression of a nucleic acid may be assessed by measuring the level of mRNA encoded by the gene, and/or by measuring the level or activity of the polypeptide encoded by the nucleic acid.

In some embodiments, host cells overexpress a polypeptide that is an activator of one or more of polypeptides of the present disclosure. Overexpression of an activator polypeptide may lead to increased abundance and activity of the polypeptide activated by the activator.

Increasing the abundance of a polypeptide of the disclosure to increase polypeptide activity may be achieved by overexpressing the polypeptide. Other methods of increasing abundance of a polypeptide are known in the art. For example, decreasing degradation of the polypeptide by cellular degradation machinery, such as the proteasome, may increase the stability and the abundance of the polypeptide. The polypeptides may be genetically modified or engineered such that they have increased resistance to cellular proteolysis, but exhibit no change in molecular activity. Polypeptides that are inhibitors of cellular factors involved in the degradation of one or more of polypeptides of the present disclosure may be introduced into host cells to increase abundance of the one or more polypeptides. Further, host cells may be treated with chemical inhibitors of the proteasome, such as cycloheximide, to increase the abundance of one or more polypeptides of the disclosure.

### Methods for Producing an Alcohol

The present disclosure relates to methods for the production of an alcohol by a host cell of the present disclosure. In certain aspects, the methods and host cells of the present disclosure relate to the production of an alcohol from an acyl-CoA. In some embodiments, the alcohol produced is a C4 alcohol.

Certain aspects of the present disclosure involve culturing a host cell of the present disclosure in a culture medium containing a suitable carbon source such that the host cell produces an alcohol. The alcohol may be a C4 alcohol such as, for example, n-butanol, crotyl alcohol, 1,3-butanediol, and/or 4-hydroxy-2-butanone. In some embodiments, a host cell of the present disclosure may contain a biosynthetic pathway for the production of n-butanol, crotyl alcohol, 1,3-butanediol, and/or 4-hydroxy-2-butanone. In some embodiments, a host cell of the present disclosure may contain a biosynthetic pathway for the production of one or more of n-butanol, crotyl alcohol, 1,3-butanediol, and 4-hydroxy-2-butanone.

Growth Conditions for Host Cells

Host cells of the present disclosure are capable of utilizing a suitable carbon source in a growth/culture medium to aid in the production of an alcohol by the host cell. “Carbon source” generally refers to a substrate or compound suitable for use as a source of carbon for cell growth. Suitable carbon sources may include, for example, glucose, glycerol, sugars, starches, and lignocellulosics, including glucose derived from cellulose and C5 sugars derived from hemicellulose, such as xylose. Additional suitable carbon sources may include, for example, various compounds such as polymers, carbohydrates, acids, alcohols, aldehydes, ketones, amino acids, peptides, etc. These include, for example, various monosaccharides, oligosaccharides, polysaccharides, a biomass polymer such as cellulose or hemicellulose, arabinose, disaccharides, such as sucrose, saturated or unsaturated fatty acids, succinate, lactate, acetate, ethanol, etc., or mixtures thereof.

In addition to an appropriate carbon source, culture media may contain suitable minerals, salts, cofactors, buffers and other components, known to those skilled in the art, suitable for the growth of the cultures and promotion of the pathways involved in the production of an alcohol. Reactions may be performed under aerobic or anaerobic conditions where aerobic, anoxic, microaerobic, or anaerobic conditions are preferred based on the requirements of the host cell.

In some embodiments, suitable carbon sources of the present disclosure may include materials derived from plant biomass. In embodiments where host cells of the present disclosure a cultured in the presence of materials derived from plant biomass, plant material may be subjected to pretreatment including ammonia fiber expansion (AFEX), steam explosion, treatment with alkaline aqueous solutions, acidic solutions, organic solvents, ionic liquids (IL), electrolyzed water, phosphoric acid, and combinations thereof. Pretreatments that remove lignin from the plant material may increase the overall amount of sugar released from the hemicellulose. Because hemicellulose degradation yields both C6 sugars (e.g., glucose) and C5 sugars (e.g., xylose) a combination of biosynthesis pathways for the production of an alcohol of the present disclosure with improved recombinant glycolysis pathways (for C6 sugar assimilation) or improved recombinant pentose phosphate pathways (for C5 sugar assimilation) may be useful for the achievement of optimal or maximal biomass utilization and yields of the alcohol.

Plant biomass suitable for use with the currently disclosed methods include various cellulose-containing materials such as, for example, Miscanthus, switchgrass, cord grass, rye grass, reed canary grass, elephant grass, common reed, wheat straw, barley straw, canola straw, oat straw, corn stover, soybean stover, oat hulls, sorghum, rice hulls, rye hulls, wheat hulls, sugarcane bagasse, copra meal, copra pellets, palm kernel meal, corn fiber, Distillers Dried Grains with Solubles (DDGS), Blue Stem, corncobs, pine wood, birch wood, willow wood, aspen wood, poplar wood, energy cane, waste paper, sawdust, forestry wastes, municipal solid waste, waste paper, crop residues, other grasses, and other woods. As described above, the plant material may require a pre-treatment to generate and/or liberate useful carbon sources such as sugars or polysaccharides. Pretreatment may involve, for example, treatment with high temperature or pressure. Such treatments are well-known to those skilled in the art.

Cofactor Specificity

Biomass degradation, and especially the degradation of hemicellulose, yields both C6 sugars such as glucose and C5 sugars such as xylose. Whereas C6 sugars are typically metabolized through the NAD⁺/NADH-dependent Embden-Meyerhof-Parnas pathway (the most common glycolytic pathway), C5 sugars are typically metabolized through the Pentose Phosphate Pathway, which is NADP⁺/NADPH-dependent. NADP⁺/NADPH-dependent enzymes of the Pentose Phosphate Pathway include a glucose dehydrogenase, such as gcd of E. coli, and a 2-keto-D-gluconate reductase, such as tiaE of E. coli. Applicants do not wish to be bound by theory. However, when host cells are used to produce an alcohol when cultured in the presence of hemicellulose-derived carbon sources, it may be beneficial to integrate NADPH-specific enzymes, such as the 3-hydroxybutyryl-CoA dehydrogenase PhaB from R. eutrophus, in the particular alcohol biosynthesis pathway to rebalance the NADP⁺ required for continued C5 sugar assimilation.

Further, because the metabolism of different carbon sources may differentially impact cellular NAD⁺/NADH- and NADP⁺/NADPH-redox systems, without wishing to be bound by theory, it is further believed that it may be beneficial to tailor certain biosynthesis pathways for the production of an alcohol to contain the most effective number of either NAD⁺/NADH-dependent or NADP⁺/NADPH-dependent enzymes. This tailoring may allow for an advantageous rebalancing of the respective redox systems and ultimately leads to more favorable or maximal carbon source utilization and yields of the alcohol. For example, when metabolizing a hexose-rich carbon source, recombinant host cells containing a greater number of NAD⁺/NADH-dependent enzymes may be used. On the contrary, when metabolizing a pentose-rich carbon source, recombinant host cells containing a greater number of NADP⁺/NADPH-dependent enzymes may be used. When metabolizing a carbon source yielding a mix of hexoses and pentoses, such as hemicellulose, recombinant host cells containing a mixture of NAD⁺/NADH-dependent and NADP⁺/NADPH-dependent enzymes may be used, such as within the recombinant n-butanol pathway.

Production of an Alcohol

The present disclosure provides methods for producing an alcohol using host cells of the present disclosure. In certain aspects, the methods and host cells of the present disclosure relate to the production of an alcohol from an acyl-CoA. In some embodiments, the alcohol produced is a C4 alcohol.

The methods and host cells of the present disclosure may be used to convert an acyl-CoA into alcohols such as, for example, a C4 alcohol. When cultured in the presence of a suitable carbon source, host cells of the present disclosure may produce, for example, one or more of n-butanol, crotyl alcohol, 1,3-butanediol, and/or 4-hydroxy-2-butanone.

In some embodiments, the production of an alcohol by a host cell such as, for example, an alcohol produced from an acyl-CoA, may be, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the production of an alcohol by a corresponding control cell. In some embodiments, the alcohol produced is a C4 alcohol. Total levels of the alcohol produced by a host cell may be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the total levels of the alcohol produced by a corresponding control cell. In some embodiments, the alcohol produced is a C4 alcohol.

In some embodiments, the production of n-butanol by a host cell may be, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the production of n-butanol by a corresponding control cell. Total levels of n-butanol produced by a host cell may be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the total levels of n-butanol produced by a corresponding control cell.

In some embodiments, the production of crotyl alcohol by a host cell may be, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the production of crotyl alcohol by a corresponding control cell. Total levels of crotyl alcohol produced by a host cell may be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the total levels of crotyl alcohol produced by a corresponding control cell.

In some embodiments, the production of 1,3-butanediol by a host cell may be, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the production of 1,3-butanediol by a corresponding control cell. Total levels of 1,3-butanediol produced by a host cell may be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the total levels of 1,3-butanediol produced by a corresponding control cell.

In some embodiments, the production of 4-hydroxy-2-butanone by a host cell may be, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the production of 4-hydroxy-2-butanone by a corresponding control cell. Total levels of 4-hydroxy-2-butanone produced by a host cell may be, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the total levels of 4-hydroxy-2-butanone produced by a corresponding control cell.

Further, the use of separate monofunctional aldehyde dehydrogenases and monofunctional alcohol dehydrogenases in host cells may allow for significant decreases in the production of ethanol in the host cell.

In some embodiments, a host cell of the present disclosure produces a non-ethanol alcohol such as, for example, a non-ethanol alcohol produced from an acyl-CoA, at concentrations that are, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the concentration of ethanol produced by the host cell. Ethanol production by a host cell that produces a non-ethanol alcohol such as, for example, a non-ethanol alcohol produced from an acyl-CoA, according to the methods of the present disclosure may be decreased by, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to a corresponding control cell. In some embodiments, the non-ethanol alcohol produced is a C4 alcohol.

In some embodiments, a host cell of the present disclosure produces n-butanol at concentrations that are, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the concentration of ethanol produced by the host cell. Ethanol production by a host cell that produces n-butanol according to the methods of the present disclosure may be decreased by, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to a corresponding control cell.

In some embodiments, a host cell of the present disclosure produces crotyl alcohol at concentrations that are, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the concentration of ethanol produced by the host cell. Ethanol production by a host cell that produces crotyl alcohol according to the methods of the present disclosure may be decreased by, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to a corresponding control cell.

In some embodiments, a host cell of the present disclosure produces 1,3-butanediol at concentrations that are, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the concentration of ethanol produced by the host cell. Ethanol production by a host cell that produces 1,3-butanediol according to the methods of the present disclosure may be decreased by, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to a corresponding control cell.

In some embodiments, a host cell of the present disclosure produces 4-hydroxy-2-butanone at concentrations that are, for example, at least 0.1-fold, at least 0.2-fold, at least 0.3-fold, at least 0.4-fold, at least 0.5-fold, at least 0.6-fold, at least 0.7-fold, at least 0.8-fold, at least 0.9-fold, at least 1-fold, at least 1.25-fold, at least 1.5-fold, at least 1.75-fold, at least 2-fold, at least 2.25-fold, at least 2.5-fold, at least 2.75-fold, at least 3-fold, at least 3.25-fold, at least 3.5-fold, at least 3.75-fold, at least 4-fold, at least 4.25-fold, at least 4.5-fold, at least 4.75-fold, at least 5-fold, at least 5.25-fold, at least 5.5-fold, at least 5.75-fold, at least 6-fold, at least 8-fold, at least 10-fold, at least 12-fold, at least 15-fold, at least 20-fold, at least 25-fold, at least 30-fold, at least 40-fold, at least 50-fold, at least 60-fold, at least 70-fold, at least 80-fold, at least 100-fold, at least 150-fold, at least 200-fold, at least 250-fold, at least 300-fold or more higher than the concentration of ethanol produced by the host cell. Ethanol production by a host cell that produces 4-hydroxy-2-butanone according to the methods of the present disclosure may be decreased by, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to a corresponding control cell.

In some embodiments where a monofunctional secondary alcohol dehydrogenase is used to modulate the production of both 4-hydroxy-2-butanone and 1,3-butanediol in a host cell, the ratio in the production titers between these two compounds may vary. For example, the ratio of production titers of 4-hydroxy-2-butanone to 1,3-butanediol may be about 1:200, about 1:150, about 1:100, about 1:75, about 1:50, about 1:25, about 1:10, about 1:5, or about 1:2.

Metabolites and products such as, for example, C4 alcohols produced by host cells according to the methods of the present disclosure may be identified and quantified using standard methods known to those of skill in the art. For example, standard methods may include standard HPLC chromatography and mass spectrometry techniques. Enzymatic activities present in or by host cells may also be analyzed and/or quantified using traditional spectrophotometric activity assays relying on the detection of NAD(P)H cofactor consumption.

Various techniques known to those of skill in the art may also be used to substantially purify an alcohol such as, for example, an alcohol produced by a host cell away from the culture medium, thus producing a substantially purified alcohol.

A substantially purified alcohol generally refers to an alcohol that is substantially free of contaminating agents (e.g. cellular material and other culture medium components) from the culture medium source where the alcohol is produced by the host cell. For example, a substantially purified alcohol may be in association with less than 30%, 20%, 10%, and more preferably 5% or less (by weight) contaminating agents. A composition containing a substantially purified alcohol preparation may include, for example, a composition where culture medium (and associated contaminating agents) represents less than about 20%, sometimes less than about 10%, and often less than about 5% of the volume of the alcohol preparation.

### EXAMPLES

The following Examples are offered for illustrative purposes and to aid one of skill in better understanding the various embodiments of the disclosure. The following Examples are not intended to limit the scope of the present disclosure in any way.

### Example 1: Monofunctional Aldehyde Dehydrogenases and Monofunctional Alcohol Dehydrogenases for Production of Fuels and Commodity Chemicals

The Example demonstrates that a monofunctional aldehyde dehydrogenase and a monofunctional alcohol dehydrogenase can be used in multiple biosynthetic pathways for the production of multiple alcohols in E. coli.

### Introduction

As described above, the major biofuel in use today is ethanol, but ethanol has major shortcomings including the low energy return compared to gasoline, high vaporizability, as well as miscibility with water. Alternative biofuels, such as n-butanol, have characteristics that are closer to current gasoline and could perform better as a replacement. Additionally, crotyl alcohol and 1,3-butanediol are commodity chemicals that could see significant application as feedstocks for butadiene used in rubber production.

A biosynthetic pathway for the production of n-butanol, crotyl alcohol, and 1,3-butanediol in E. coli have been previously developed. Applicants sought to explore methods of recalibrating these pathways to improve the production of these commodity chemicals in host cells.

### Materials and Methods

Commercial Materials

Luria-Bertani (LB) Broth Miller, LB Agar Miller, and Terrific Broth (TB) were purchased from EMD Biosciences (Darmstadt, Germany). Carbenicillin (Cb), isopropyl-β-D-thiogalactopyranoside (IPTG), phenylmethanesulfonyl fluoride (PMSF), tris(hydroxymethyl)aminomethane hydrochloride (Tris-HCl), sodium chloride, dithiothreitol (DTT), kanamycin (Km), ethyl acetate and ethylene diamine tetraacetic acid disodium dihydrate (EDTA), were purchased from Fisher Scientific (Pittsburgh, Pa.). Coenzyme A trilithium salt (CoA), acetyl-CoA, nicotinamide adenine dinucleotide reduced form dipotassium salt (NADH), β-mercaptoethanol, sodium phosphate dibasic hepthydrate, and N,N,N′,N′-tetramethyl-ethane-1,2-diamine (TEMED) were purchased from Sigma-Aldrich (St. Louis, Mo.). Acrylamide/Bis-acrylamide (30%, 37.5:1), electrophoresis grade sodium dodecyl sulfate (SDS), Bio-Rad protein assay dye reagent concentrate and ammonium persulfate were purchased from Bio-Rad Laboratories (Hercules, Calif.). Restriction enzymes, T4 DNA ligase, Phusion DNA polymerase, T5 exonuclease, and Taq DNA ligase were purchased from New England Biolabs (Ipswich, Mass.). Deoxynucleotides (dNTPs) and Platinum Taq High-Fidelity polymerase (Pt Taq HF) were purchased from Invitrogen (Carlsbad, Calif.). PageRuler™ Plus prestained protein ladder was purchased from Fermentas (Glen Burnie, Md.). Oligonucleotides were purchased from Integrated DNA Technologies (Coralville, Iowa), resuspended at a stock concentration of 100 μM in 10 mM Tris-HCl, pH 8.5, and stored at either 4° C. for immediate use or −20° C. for longer term use. DNA purification kits and Ni-NTA agarose were purchased from Qiagen (Valencia, Calif.). Amicon Ultra 10,000 centrifugal concentrators were purchased from EMD Millipore (Billerica, Mass.).

Bacterial Strains

E. coli DH10B-T1^{R} and BL21(de3)T1^{R} were used for DNA construction and heterologous protein production, respectively. Strains, plasmids, and oligonucleotides used herein are described in detail in Table 1, Table 2, and Table 3.

Gene Naming Conventions

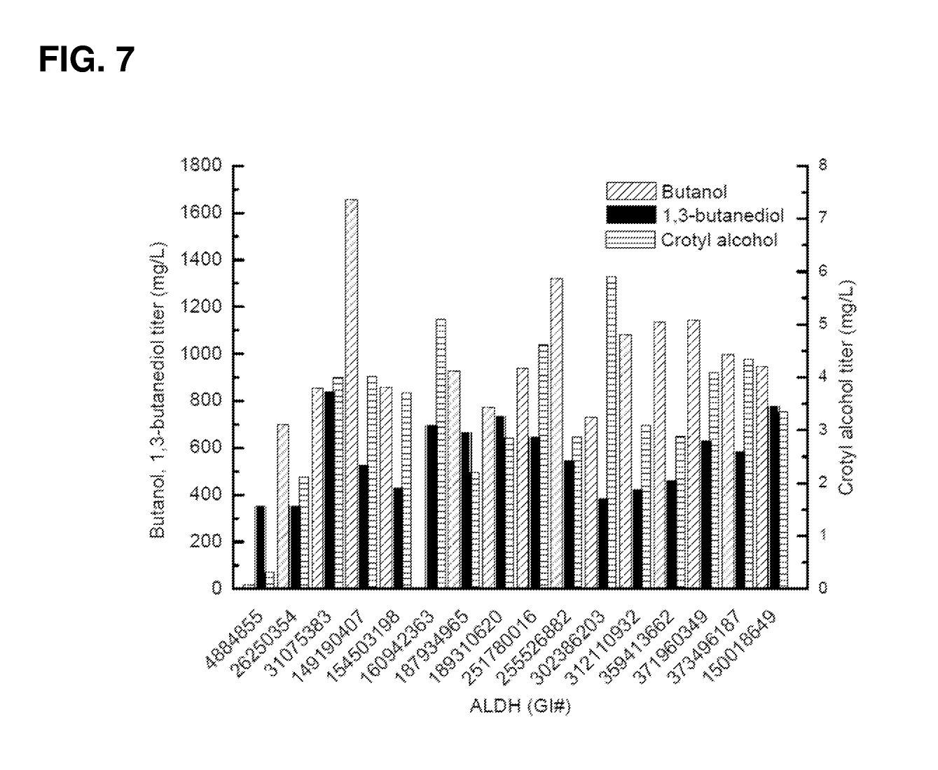

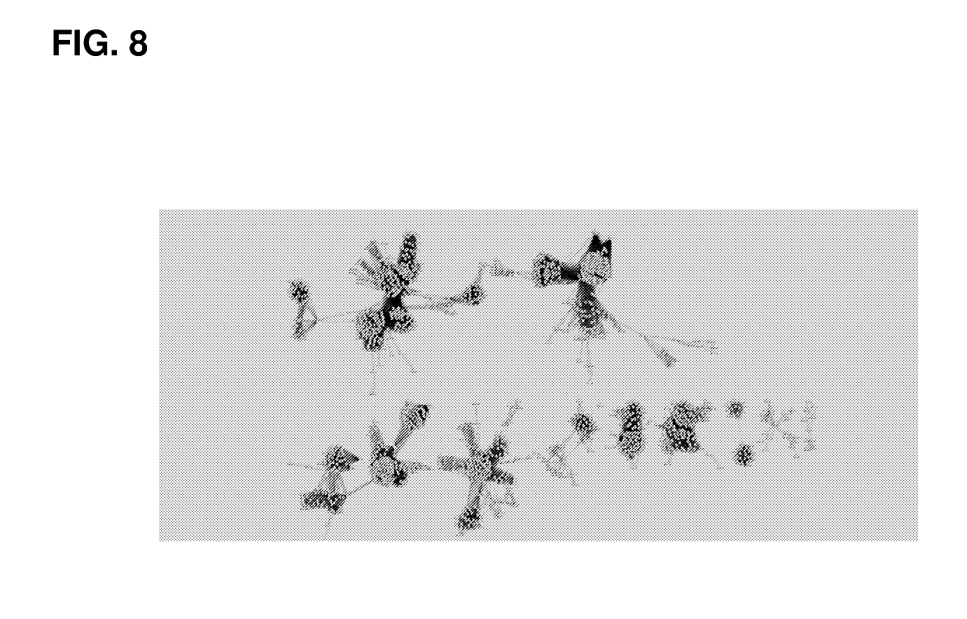

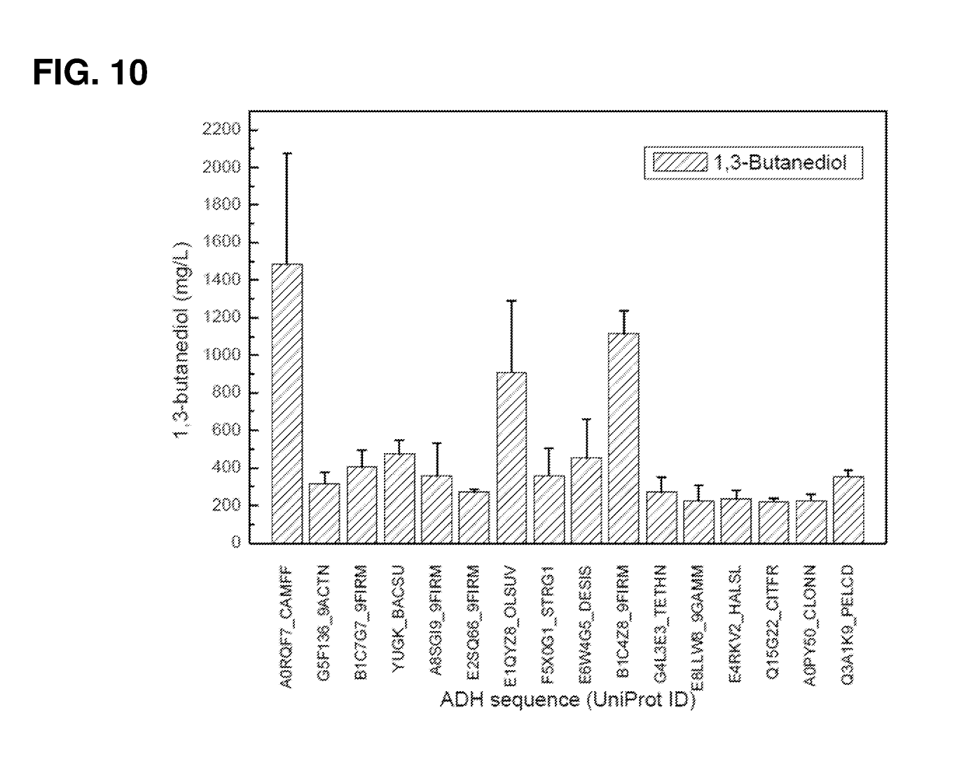

ALDH genes referred to throughout this Example are named according to the order of their appearance on the x-axis in FIG. 7. For example, GI 4884855 is also referred to as ALDH1 (first gene on the x-axis), and GI 150018649 is also referred to as ALDH16 (sixteenth gene on the x-axis). Similarly, ADH genes referred to throughout this Example are named according to the order of their appearance on the x-axis in FIG. 10. For example, A0RQF7_CAMFF is also referred to as ADH1 (first gene on the x-axis), and GI Q3A1K9_PELCD is also referred to as ADH16 (sixteenth gene on the x-axis).

Gene and Plasmid Construction

Gibson assembly was used to carry out plasmid construction. All PCR amplifications were carried out with Phusion or Platinum Taq High Fidelity DNA polymerases. All constructs were verified by sequencing (Quintara Biosciences; Berkeley, Calif.).

pET23a-His₆TEV-aldh3.

Aldh3 was amplified from pCDF3-aldh3 using the primers HisTev_aldh3 GF1 and HisTev_aldh3 GR1 and inserted into the SfoI-XhoI restriction sites of pET23a to generate pET23a-His₆TEV_aldh3.

pET23a-His₆TEV-aldh16.

Aldh16 was amplified from pCDF3-aldh16 using the primers HisTev_aldh16 GF1 and HisTev_aldh16 GR1 and inserted into the SfoI-XhoI restriction sites of pET23a to generate pET23a-His₆TEV_aldh16.

pCWori-strep_aldh3.

Aldh3 was amplified from pCDF3-aldh3 using the primers strep_aldh3 GF1 and aldh3 GR1 and inserted into the NdeI-HindIII restriction sites of pCWori to generate pCWori-strep_aldh3.

pCWori-strep_aldh16.

Aldh16 was amplified from pCDF3-aldh16 using the primers strep_aldh16 GF1 and aldh16 GR1 and inserted into the NdeI-HindIII restriction sites of pCWori to generate pCWori-strep_aldh16.

pT533-phaA.phaB.

The oligos trc.crt delete GO1 and trc.crt delete GO2 were inserted into the XbaI restriction site of pT5T33-phaA.phaB-crt to generate pT533-phaA.phaB.

pT533-phaA.HBD.

The oligos trc.crt delete GO1 and trc.crt delete GO2 were inserted into the XbaI restriction site of pT5T33-phaA.HBD-crt to generate pT533-phaA.HBD.

pCDF3-ter.aldh1-16.

Different pCDF3-ter plasmids were constructed that contained an alcohol dehydrogenase (ALDH). As 16 different ALDHs were tested, 16 different plasmids, each with a different ALDH, were constructed.

pCDF3-aldh1-16.

The oligos ter delete GO5 and ter delete GO6 were inserted into the BamHI-EcoRI restriction sites of pCDF3-ter.aldh1-16 to generate pCDF3-aldh1-16.

Bioinformatics Search for Aldehyde Dehydrogenases

A bioinformatics search was conducted to identity putative aldehyde dehydrogenases.

Bioinformatics Search for Alcohol Dehydrogenases

The Fe-ADH sequence family (PF00465) was filtered using cd-hit (http://www.bioinformatics.org/cd-hit/) to remove sequences greater than 90% identical. The remaining sequences were blasted all-vs-all using BLAST and the resulting sequence similarity network was visualized in Cytoscape at various E-value cutoffs. Alcohol dehydrogenases of known substrate specificity were overlaid on the network and sequences were randomly sampled from adjacent sequence clusters.

Expression of His-Tagged Proteins

TB (1 L) containing carbenicillin (50 μg/mL) in a 2.8 L Fernbach baffled shake flask was inoculated to OD₆₀₀=0.05 with an overnight TB culture of freshly transformed E. coli containing the appropriate overexpression plasmid. The cultures were grown at 37° C. at 200 rpm to OD₆₀₀=0.6 to 0.8 at which point cultures were cooled on ice for 20 min, followed by induction of protein expression with 1 mM IPTG and overnight growth at 16° C. Cell pellets were harvested by centrifugation at 9,800×g for 7 min and resuspended at 20 mL/L of culture with Buffer A (50 mM sodium phosphate, 300 mM sodium chloride, 20 mM imidazole, 0.5 mM EDTA, pH 8.0) supplemented with 2 mg/mL lysozyme and 2 μL/50 mL final volume Benzonase and frozen at −80° C.

Purification of His-Tagged Proteins

Frozen cell suspensions were thawed and frozen twice before finally thawing and adding 0.5 mM PMSF as a 50 mM stock solution in ethanol dropwise. The cell suspension was lysed at with a Misonix 3000 probe sonicator at full power with a 15 second on, 60 second off cycle for a total sonication time of 2.5 minutes. The lysate was centrifuged at 15,300×g for 20 min at 4° C. to separate the soluble and insoluble fractions. DNA was precipitated in the soluble fraction by addition of 1% streptomycin sulfate as a 20% w/v stock solution added dropwise. The precipitated DNA was removed by centrifugation at 15,300×g for 20 min at 4° C. The lysate was loaded onto a Ni-NTA agarose column (Qiagen, 1 mL resin/L expression culture) by gravity flow. The column was washed with 20 column volumes Buffer A. The protein was then eluted with 250 mM imidazole in Buffer A.

Fractions containing the target protein were pooled by A_{280 nm} and supplemented with 100 mM DTT to 1 mM final. TEV protease (QB3 Macrolab) was added at a 1:20 ratio w/w. Protein was then placed in 10 kDa MWCO dialysis tubing in 1.8 L Buffer A with 1 mM DTT and dialyzed overnight at 4° C.

Dialyzed protein was loaded onto the previous Ni-NTA agarose column equilibrated with Buffer A and the flowthrough was collected. This procedure was repeated two times and the column was washed with 1 column volume of buffer A. The pooled flowthrough was concentrated in an Amicon Ultra 10,000 MWCO concentrator to a final volume of 2 mL. Concentrated protein was loaded on a Superdex 200 SEC column (GE Healthcare; Piscataway, N.J.) connected to an ÄKTApurifier FPLC (1 mL/min; GE Healthcare). Fractions containing ALDH protein by A₂₈₀ were pooled and concentrated in an Amicon Ultra 10,000 MWCO concentrator. Concentrated protein was supplemented with glycerol to 10% v/v and stored at −80° C.

Crystallization and Structure Determination of GA-ALDH3 and GA-ALDH16

Protein crystals were obtained using the sitting drop vapor diffusion method by combining equal volumes of a 10 mg/mL protein solution and a reservoir solution [0.2 M tri-sodium citrate (pH 7.5) and 20% (w/v) polyethylene glycol 3350]. Crystals grew within 2 days and were cryoprotected by being briefly soaked in a solution containing 75% reservoir solution and 25% ethylene glycol followed by flash-freezing in liquid nitrogen. Data were collected at Beamline 8.3.1 at the Advanced Light Source (Lawrence Berkeley National Laboratory, Berkeley, Calif.). Data sets for native crystals were collected at a wavelength of 1.116 Å. Data sets were processed and merged with XDS and XSCALE. Phases were determined by molecular replacement using Phenix AutoMR and AutoBuild to build a near-complete chain trace of each crystal. Iterative cycles of Phenix AutoRefine and manual refinement in Coot32 were used to generate the final model.

Expression of Strep-Tagged Proteins

TB (1 L) containing carbenicillin (50 μg/mL) in a 2.8 L Fernbach baffled shake flask was inoculated to OD₆₀₀=0.05 with an overnight TB culture of freshly transformed E. coli containing the appropriate overexpression plasmid. The cultures were grown at 37° C. at 200 rpm to OD₆₀₀=0.6 to 0.8 at which point cultures were cooled on ice for 20 min, followed by induction of protein expression with 1 mM IPTG and overnight growth at 16° C. Cell pellets were harvested by centrifugation at 9,800×g for 7 min and resuspended at 20 mL/L of culture with Buffer W (100 mM Tris-HCl, 150 mM sodium chloride, 1 mM EDTA, pH 8.0) supplemented with 2 mg/mL lysozyme and 2 μL/50 mL final volume Benzonase and frozen at −80° C.

Purification of Strep-Tagged Proteins

Frozen cell suspensions were thawed and frozen twice before finally thawing and adding 0.5 mM PMSF as a 50 mM stock solution in ethanol dropwise. The cell suspension was lysed at with a Misonix 3000 probe sonicator at full power with a 15 second on, 60 second off cycle for a total sonication time of 2.5 minutes. The lysate was centrifuged at 15,300×g for 20 min at 4° C. to separate the soluble and insoluble fractions. DNA was precipitated in the soluble fraction by addition of 0.5% polyethylenimine as a 15% v/v stock solution added dropwise. The precipitated DNA was removed by centrifugation at 15,300×g for 20 min at 4° C. The lysate was loaded onto a Strep-tactin Superflow High Capacity column (IBA, 1 mL resin/L expression culture) by gravity flow. The column was washed with 20 column volumes Buffer W. The protein was then eluted with 2.5 mM desthiobiotin in Buffer W. Fractions containing ALDH protein by A₂₈₀ were pooled and concentrated in an Amicon Ultra 10,000 MWCO concentrator. Concentrated protein was supplemented with glycerol to 10% v/v and stored at −80° C.

Enzyme Assays

Activity of ALDH proteins was measured by monitoring the oxidation of NADH at 340 nm at 30° C. The assay mixture (400 μL) contained 100 μM NADH in 100 mM Tris 1 mM DTT pH 7.5. The reaction was initiated by the addition of substrate. Kinetic parameters (k_{cat}, K_{M}) were determined by fitting the data using Microcal Origin to the equation: vₒ=vₘₐₓ [S]/(K_{M}+[S]), where v is the initial rate and [S] is the substrate concentration. Data are reported as mean±s.e. (n=3) unless otherwise noted with standard error derived from the nonlinear curve fitting. Error bars on graphs represent mean±s.d. (n=3). Error in k_{cat}/K_{M} is calculated by propagation of error from the individual kinetic parameters.

Synthesis of (S)- and (R)-3-hydroxybutyryl-CoA

His6-Hbd (35 μg/ml) or His6-PhaB (17.5 μg/ml) was incubated with acetoacetyl-CoA (12.5 mM) and NADH or NADPH (125 mM) in 100 mM Tris-HCl, pH 7.5 (300 μL) for 60 min at 30° C. to produce (5)- and (R)-3-hydroxybutyryl-CoA, respectively. Both products were isolated by RP-HPLC using an Eclipse XDB C18 column (5 μm, 9.4×250 mm, Agilent) using a 0-100% acetonitrile gradient over 25 min with 10 mM acetic acid, pH 4.0 as the mobile phase. The (S)-3-hydroxybutyryl-CoA was further purified by RP-HPLC using an Eclipse XDB C-8 column (3.5 μm, 3.0×150 mm, Agilent) using a 0-100% acetonitrile gradient over 25 min with 20 mM triethylamine, 10 mM acetic acid, pH 4.0 as the mobile phase. Purified (S)- and (R)-3-hydroxybutyryl-CoA were lyophilized following each purification step and analyzed by LC-MS using an Eclipse XDB C18 column (5 μm, 4.6×150 mm, Agilent) using a 0-100% acetonitrile gradient over 25 min with 10 mM acetic acid, pH 4.0 as the mobile phase. ESIMS (M-H) calculated for C25H41O18N7P3S m/z, 852.1, found 852.1 ((S)-3-hydroxybutyryl-CoA) and 852.1 ((R)-3-hydroxybutyryl-CoA).

Cell Culture

E. coli strains were transformed by electroporation using the appropriate plasmids. A single colony from a fresh transformation was then used to seed an overnight culture grown in Terrific Broth (TB) (EMD Biosciences) supplemented with 1.5% (w/v) glucose and appropriate antibiotics at 37° C. in a rotary shaker (200 r.p.m.). Antibiotics were used at a concentration of 50 μg ml⁻¹ for strains with a single resistance marker. For strains with multiple resistance markers, kanamycin and chloramphenicol were used at 25 μg ml⁻¹ and carbenicillin was used at 50 μg ml⁻¹.

In Vivo Production of Butanol, 1,3-Butanediol, Crotyl Alcohol, and 4-Hydroxy-2-Butanone

Overnight cultures of freshly transformed E. coli strains were grown for 12-16 h in TB at 37° C. and used to inoculate TB (50 ml) with glucose replacing the standard glycerol supplement (1.5% (w/v)) glucose for aerobic cultures and 2.5% (w/v) glucose for anaerobic cultures) and appropriate antibiotics to an optical density at 600 nm (OD₆₀₀) of 0.05 in a 250 mL-baffled flask or a 250 mL-baffled anaerobic flask. The cultures were grown at 37° C. in a rotary shaker (200 r.p.m.) and induced with IPTG (1.0 mM) at OD₆₀₀=0.35-0.45. At this time, the growth temperature was reduced to 30° C., and the culture flasks were sealed with Parafilm M (Pechiney Plastic Packaging) to prevent product evaporation for aerobic cultures. Anaerobic cultures were sealed and the headspace was sparged with argon for 5 minutes immediately follow induction. Aerobic cultures were unsealed for 10 to 30 min every 24 h then resealed with Parafilm M, and additional glucose (1% (w/v)) was added 1 day post-induction. Samples were quantified after 3 d of cell culture.

Quantification of n-Butanol

Samples (2 ml) were removed from cell culture and cleared of biomass by centrifugation at 20,817 g for 2 min using an Eppendorf 5417R centrifuge. The supernatant or cleared medium sample was then mixed in a 9:1 ratio with an aqueous solution containing the isobutanol internal standard (10,000 mg l⁻¹). These samples were then analyzed on a Trace GC Ultra (Thermo Scientific) using an HP-5MS column (0.25 mm×30 m, 0.25 μM film thickness, J & W Scientific). The oven program was as follows: 75° C. for 3 min, ramp to 300° C. at 45° C. min⁻¹, 300° C. for 1 min. n-Butanol was quantified by flame ionization detection (FID) (flow: 350 ml min⁻¹ air, 35 ml min⁻¹ H2 and 30 ml min⁻¹ helium). Samples containing n-butanol levels below 500 mg 1⁻¹ were requantified after extraction of the cleared medium sample or standard (500 μl) with toluene (500 μl) containing the isobutanol internal standard (100 mg 1⁻¹) using a Digital Vortex Mixer (Fisher) for 5 min set at 2,000. The organic layer was then quantified using the same GC parameters with a DSQII single-quadrupole mass spectrometer (Thermo Scientific) using single-ion monitoring (m/z 41 and 56) concurrent with full scan mode (m/z 35-80). Samples were quantified relative to a standard curve of 2, 4, 8, 16, 31, 63, 125, 250, 500 mg 1⁻¹ n-butanol for MS detection or 125, 250, 500, 1,000, 2,000, 4,000, 8,000 mg 1⁻¹ n-butanol for FID detection. Standard curves were prepared freshly during each run and normalized for injection volume using the internal isobutanol standard (100 or 1,000 mg 1⁻¹ for MS and FID, respectively).

Quantification of Crotyl Alcohol

Samples (2 ml) were removed from cell culture and cleared of biomass by centrifugation at 20,817 g for 2 min using an Eppendorf 5417R centrifuge. The cleared medium sample or standard (500 μl) was extracted with toluene (500 μl) containing the isobutanol internal standard (100 mg 1⁻¹) using a Digital Vortex Mixer (Fisher) for 5 min set at 2,000. The organic layer was then analyzed on a Trace GC Ultra (Thermo Scientific) using an HP-5MS column (0.25 mm×30 m, 0.25 μM film thickness, J & W Scientific). The oven program was as follows: 75° C. for 4 min, ramp to 300° C. at 45° C. min⁻¹, 300° C. for 2 min. Crotyl alcohol was detected with a DSQII single-quadrupole mass spectrometer (Thermo Scientific) using single-ion monitoring (m/z 29, 41, 43, and 57) concurrent with full scan mode (m/z 37-58). Samples were quantified relative to a standard curve of 2, 4, 8, 16, 31, 63, 125, 250, 500 mg 1⁻¹ crotyl alcohol for MS detection. Standard curves were prepared freshly during each run and normalized for injection volume using the internal isobutanol standard (100 mg l⁻¹).

Quantification of 1,3-Butanediol

Samples (2 ml) were removed from cell culture and cleared of biomass by centrifugation at 20,817 g for 2 min using an Eppendorf 5417R centrifuge. The cleared medium samples, or standards prepared in TB medium, were diluted 1:100 into water and filtered through a 0.22 μm filter (EMD Millipore MSGVN2210). The samples were analyzed on an Agilent 1290 HPLC (Agilent) using a Rezex ROA-Organic Acid H+(8%) column (150×4.6 mm, Phenomenex) with isocratic elution using 0.5% formic acid (0.3 mL/min, 55° C.). Samples were detected with an Agilent 6460C triple quadrupole MS with Jet Stream ESI source (Agilent), operating in positive MRM mode (91-73 transition, fragmentor 50 V, collision energy 0 V, cell accelerator voltage 7 V, delta EMV+400). Samples were quantified relative to a standard curve of 31, 63, 125, 250, 500, 1000, 2000, 4000 mg l⁻¹ 1,3-butanediol.

Quantification of 4-Hydroxy-2-Butanone

Samples (2 ml) were removed from cell culture and cleared of biomass by centrifugation at 20,817 g for 2 min using an Eppendorf 5417R centrifuge. The cleared medium samples, or standards prepared in TB medium, were diluted 1:100 into water and filtered through a 0.22 μm filter (EMD Millipore MSGVN2210). The samples were analyzed on an Agilent 1290 HPLC (Agilent) using a Rezex ROA-Organic Acid H+(8%) column (150×4.6 mm, Phenomenex) with isocratic elution using 0.5% formic acid (0.3 mL/min, 55° C.). Samples were detected with an Agilent 6460C triple quadrupole MS with Jet Stream ESI source (Agilent), operating in positive MRM mode (89-71 transition, fragmentor 50 V, collision energy 0 V, cell accelerator voltage 7 V, delta EMV+400). Samples were quantified relative to a standard curve of 31, 63, 125, 250, 500, 1000, 2000, 4000 mg l⁻¹ 1,3-butanediol.

TABLE 1
Strains

| Strain | Genotype |
|---|---|
| BL21(de3) | F⁻ ompT gal dcm lon hsdS_{B}(r_{B}⁻ m_{B}⁻) λ(DE3 |
| | [lacI lacUV5-T7 gene 1 ind1 sam7 nin5]) |
| DH1 | endA1 recA1 gyrA96 thi-1 glnV44 relA1 hsdR17(rK⁻ |
| | mK⁺) λ⁻ |
| MC1.24 | endA1 recA1 gyrA96 thi-1 glnV44 relA1 hsdR17(rK⁻ |
| | mK⁺) λ⁻ ΔadhE ΔldhA Δack-pta ΔpoxB |
| | ΔfrdBC |

TABLE 2
Plasmids

| Plasmid | Description |
|---|---|
| pET23a-His₆TEV-aldh3 | his₆TEV-aldh3 (T7), lacI, Cb^{r}, ColE1 |
| pET23a-His₆TEV-aldh16 | his₆TEV-aldh16 (T7), lacI, Cb^{r}, ColE1 |
| pCWori-Strep_aldh3 | strep-aldh3 (double Tac), lacI, Cb^{R}, ColE1 |
| pCWori-Strep_aldh16 | strep-aldh3 (double Tac), lacI, Cb^{R}, ColE1 |
| pT533-phaA.phaB | phaA.phaB (T5), lacIq, Cm^{R}, p15a |
| pT533-phaA.HBD | phaA.phaB (T5), lacIq, Cm^{R}, p15a |
| pCDF3-ter.aldh1-16 | ter.aldh1-16 (double Tac), lacIq, Sp^{R}, |
| | CloDF13cop3 |
| pCDF3-aldh1-16 | aldh1-16 (double Tac), lacI, Sp^{R}, |
| | CloDF13cop3 |
| pCWO.trc-ter-aldh16.adh1-14 | ter (double Tac), aldh16.adh1-14 (Trc), |
| | lacIq, Cb^{R}, ColE1 |
| pCWO.trc-ter-aldh16.dhaT1-8 | ter (double Tac), aldh16.dhaT1-8 (Trc), |
| | lacIq, Cb^{R}, ColE1 |

TABLE 3
Oligonucleotide Sequences

| Name | SEQ ID No. | Sequence |
|---|---|---|
| trc.crt delete GO1 | 267 | caagcttgcatgcctgcaggtcgactctagattagcccatgtgcaggccaccgttcaggg |
| trc.crt delete GO2 | 268 | gaacggtggcctgcacatgggctaatctagagtcgacctgcaggcatgcaagcttggctg |
| phaB SF1 | 269 | gtgcatggctgtcttccg |
| HBD SF1 | 270 | gcacacgctgctgaaaaag |
| aldh3 SF1 | 271 | acgcaattatcaaacacccgtcc |
| aldh6 SF1 | 272 | ggaagagccgtctattgagaacac |
| aldh7 SF1 | 273 | gcacccgtacatcaagctgc |
| HisTev_aldh16 GF1 | 274 | tcatcatgagaatctctacttccagggtaccggcgccatgaataaagacaccctgattcc |
| HisTev_aldh16 GR1 | 275 | gttagcagccggatctcagtggtggtggtggtggtgctcgagtttagccggccagaacac |
| HisTev_aldh3 GF1 | 276 | catcatgagaatctctacttccagggtaccggcgccatgattaaggacactctcgtaagc |
| HisTev_aldh3 GR1 | 277 | agcagccggatctcagtggtggtggtggtggtgctcgagtttaacccgccagaacacaac |
| HisTev_aldh6 GF1 | 278 | catcatcatgagaatctctacttccagggtaccggcgccatgaaagagggtgtaattcgc |
| HisTev_aldh6 GR1 | 279 | tagcagccggatctcagtggtggtggtggtggtgctcgagtttaacgaatgctaaaggcg |
| HisTev_aldh7 GF1 | 280 | catcatcatcatgagaatctctacttccagggtaccggcgccatggaacgcaacttgtcg |
| HisTev_aldh7 GR1 | 281 | agcagccggatctcagtggtggtggtggtggtgctcgagtttaaccggccagaacgcaac |
| ter delete GO5 | 282 | agcggataacaatttcacacaggaaacaggatccgaattcaaaaaaggaggtaaaaaatg |
| ter delete GO6 | 283 | cattttttacctccttttttgaattcggatcctgtttcctgtgtgaaattgttatccgct |
| aldh3 GR1 | 284 | actttgaaccacagcattaggacctcctctggtaagctctagattaacccgccagaacac |
| aldh6 GR1 | 285 | tttgaaccacagcattaggacctcctctggtaagctctagattaacgaatgctaaaggcg |
| aldh7 GR1 | 286 | actttgaaccacagcattaggacctcctctggtaagctctagattaaccggccagaacgc |
| aldh16 GF4 | 287 | gaagataagattctgaaacatgagc |
| aldh16.yqhD GR1 | 288 | agattaaagttgttcatctttacctcctgatagaagtctcgagttagccggccagaacac |
| aldh16.yqhD GF1 | 289 | tggccggctaactcgagacttctatcaggaggtaaagatgaacaactttaatctgcacac |
| yqhD GR2 | 290 | tcatgtttgacagcttatcatcgataagcttgagctcttagcgggcggcttcgtatatac |
| aldh16.fucO GR1 | 291 | atcattctgttagccattgtctccccccctgcgccggctcgagttagccggccagaacac |
| aldh16.fucO GF1 | 292 | ggccggctaactcgagccggcgcagggggggagacaatggctaacagaatgattctgaac |
| fucO GR2 | 293 | gctcatgtttgacagcttatcatcgataagcttgagctcttaccaggcggtatggtaaag |
| aldh16.dhaT GR1 | 294 | ttagccggccagaacac |
| yqhD GF1 | 295 | gtaacttcacgcgccaacgtcgttgtgttctggccggctaactcgagacttctatcaggaggtaaa |
| | | gatgaacaactttaatctgcacac |
| rrnB-1 GF1 | 296 | ggtattaactacgaggcagaagttg |
| rrnB-1 GR1 | 297 | gttccctactctcgcatgggCTgaccccacactaccatcg |
| rrnB-2 GF1 | 298 | cgatggtagtgtggggtcAGcccatgcgagagtagggaac |
| rrnB-2 GR1 | 299 | cagcttccgatggctgcc |
| BsaI delete GO1 | 300 | ctgataaatctggagccggtgagcgtgggtGAcgcggtatcattgcagcactggggccag |
| BsaI delta GO2 | 301 | ctggccccagtgctgcaatgataccgcgTCacccacgctcaccggctccagatttatcag |

### Results

Efforts to Maximize Alcohol Production

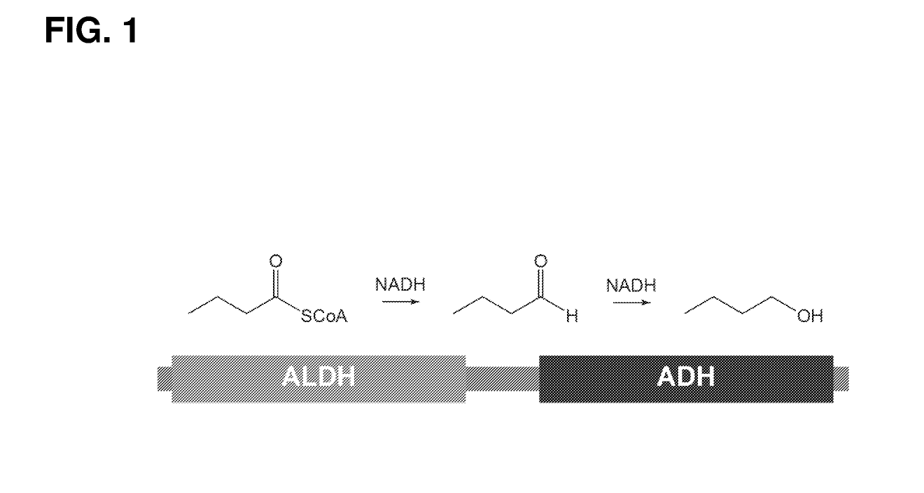

While developing improved or more efficient biosynthetic pathways for the production of n-butanol, crotyl alcohol, and 1,3-butanediol, it was discovered that the final enzyme in each pathway, AdhE2, was catalyzing significant production of the undesired side product ethanol. Formation of this side product derives from a lack of substrate specificity by AdhE2 for C4 substrates to the exclusion of smaller substrates. AdhE2 is an enzyme that acts as a bifunctional alcohol and aldehyde dehydrogenase. AdhE2 reduces acyl-CoAs to aldehydes via the aldehyde dehydrogenase (ALDH) domain, and aldehydes are subsequently reduced to alcohols via the alcohol dehydrogenase (ADH) domain. Without wishing to be bound by theory, it is thought that the mechanism of reduction involves substrate channeling of the volatile and reactive aldehyde intermediate between the two linked domains of the enzyme (FIG. 1). Further, and without wishing to be bound by theory, it is thought that this substrate channeling mechanism both shields the cell from a toxic intermediate as well as ensuring efficient carbon flow through a high-flux fermentation pathway. However, as discussed above, AdhE2 allows for significant production of the undesired ethanol side product. Applicants sought to explore ways to minimize production of the ethanol side product, as any ethanol produced decreases the yield of the desired product and increases downstream separation costs.

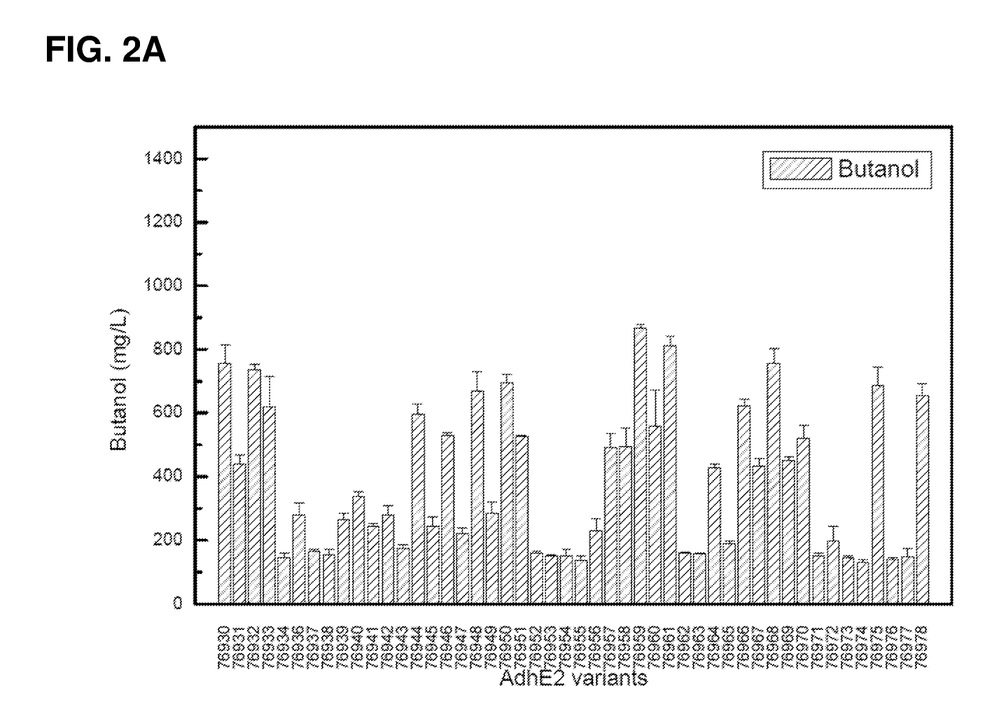

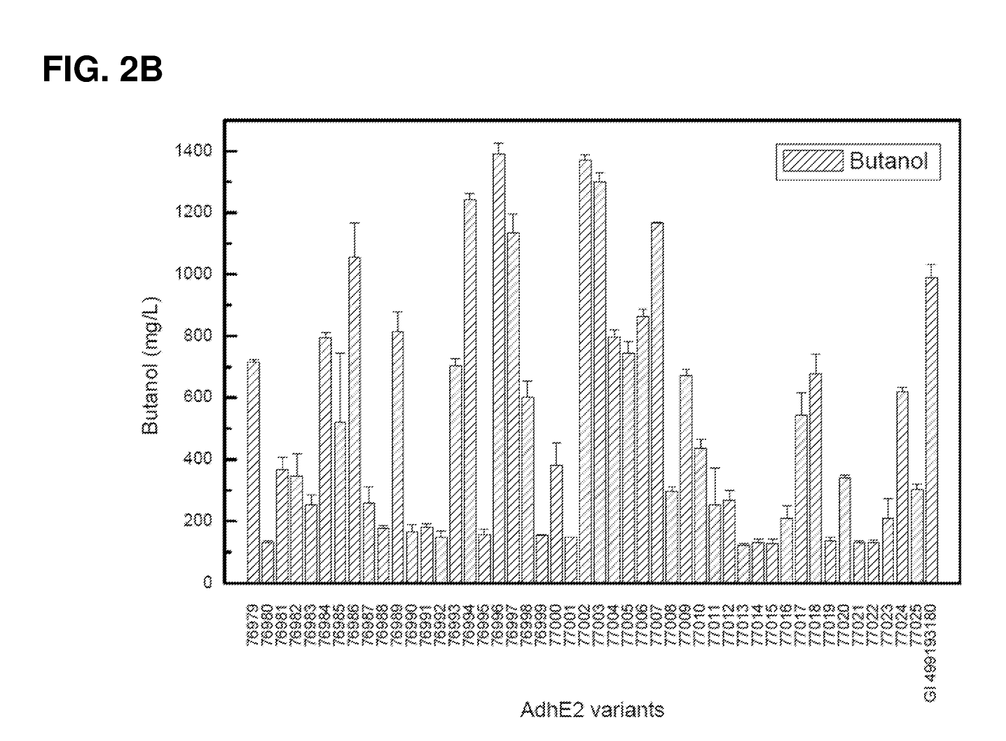

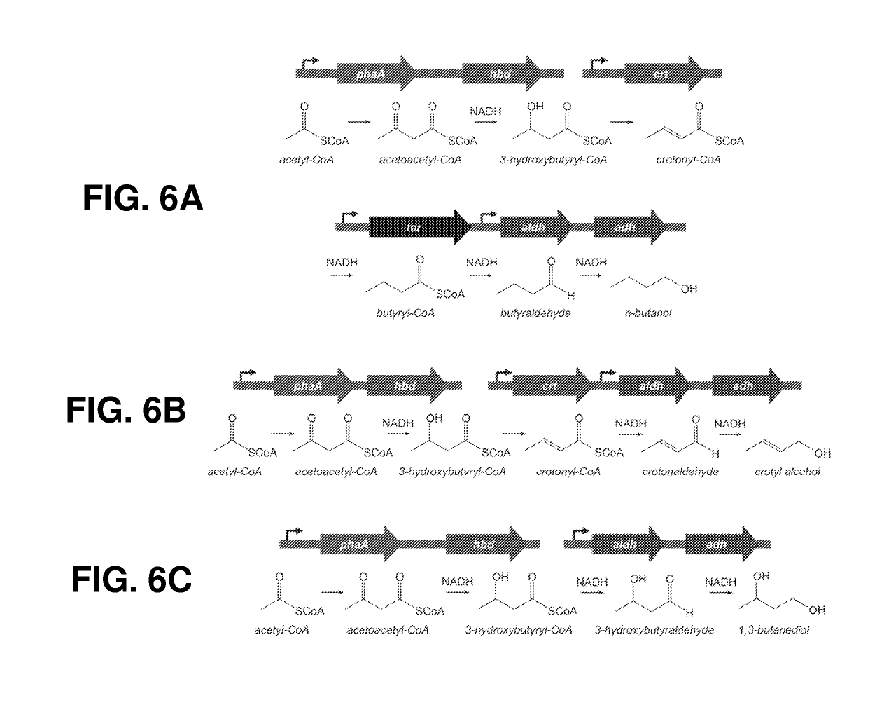

To maximize AdhE2 for C4 substrate specificity, a phylogenetics-informed mutagenesis strategy was used in an attempt to modify the substrate preference of AdhE2 for C4 substrates. E. coli cells were genetically engineered to contain an n-butanol biosynthetic pathway (FIG. 6A) using either WT AdhE2 or a particular AdhE2 variant subjected to mutagenesis according to the phylogenetics-informed mutagenesis strategy. The AdhE2 variants contained targeted amino acid substitutions. However, this approach proved ineffective, as no variant produced a significantly better butanol:ethanol ratio in the host cells (FIG. 2A and FIG. 2B).

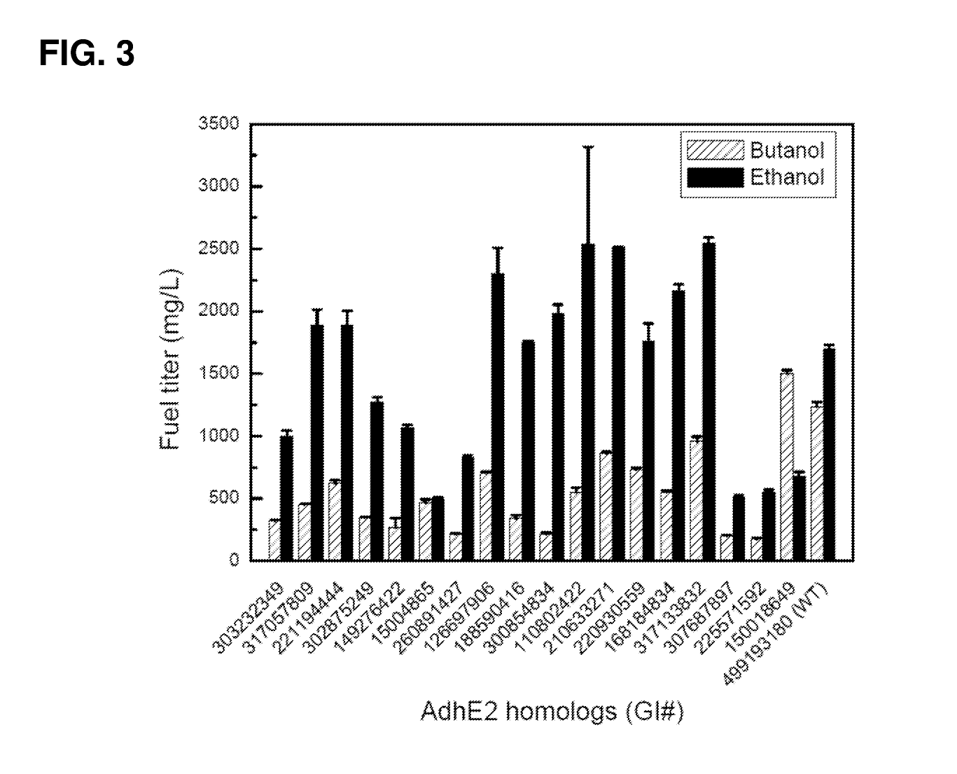

Further, a phylogenetic search for members of the AdhE2 superfamily was conducted to select sequences that would preferentially act on C4 substrates. E. coli cells were genetically engineered to contain an n-butanol biosynthetic pathway (FIG. 6A) and containing AdhE2 (WT) or various AdhE2 homologs (AdhE2 homolog GI numbers on x-axis of FIG. 3). Surprisingly, no members of the bifunctional AdhE2 family showed improved C4 substrate specificity (FIG. 3).

However, through the process of screening for improved AdhE2 sequences, it was discovered that independent monofunctional enzymes could robustly enable production of butanol while largely eliminating undesirable ethanol production seen from AdhE2. As can be seen in FIG. 3, only E. coli expressing a monofunctional aldehyde dehydrogenase (GI 150018649) produce a favorable butanol:ethanol ratio. This result was unexpected because, without wishing to be bound by theory, it is thought that independent monofunctional aldehyde and alcohol dehydrogenases require the reactive and volatile aldehyde substrate to freely diffuse from one enzyme to the next instead of the direct channeling mechanism likely employed by the bifunctional AdhE2. Prior to this discovery, it was thought that a high-flux fermentation pathway that released a volatile or reactive intermediate would both limit the yield of the pathway and prove toxic to the host cell. However, this result was not observed, and further, this split aldehyde dehydrogenase and alcohol dehydrogenase approach has the added benefit of enabling combinations of aldehyde and alcohol dehydrogenases tailored for the production of each product.

Exploring Monofunctional Aldehyde and Alcohol Dehydrogenases in Alcohol Production

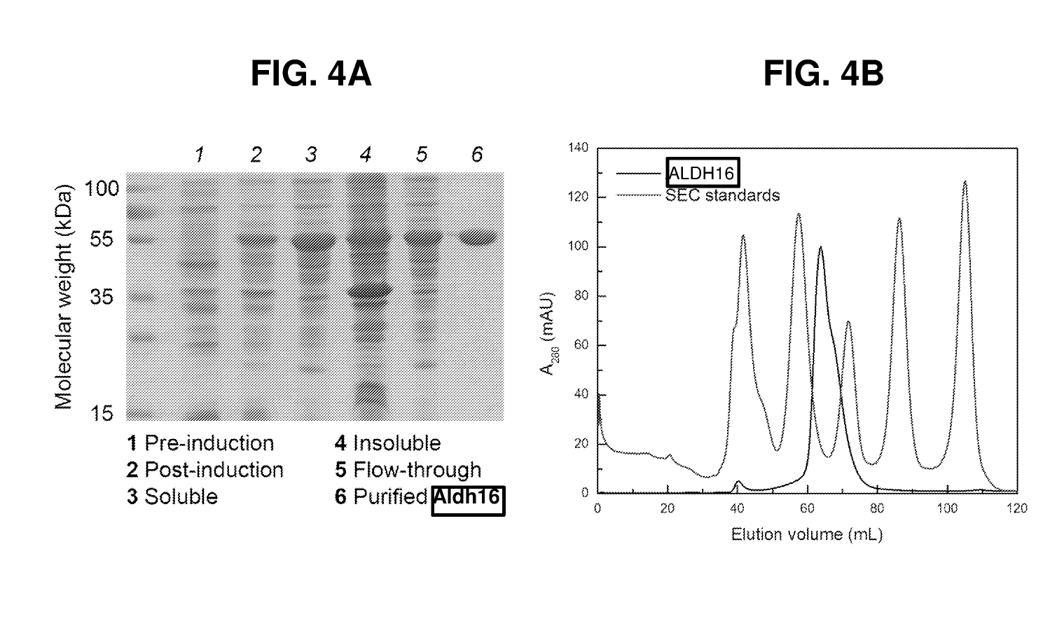

To further explore the potential for expressing monofunctional aldehyde and alcohol dehydrogenases in host cells for the production of alcohols, various aldehyde and alcohol dehydrogenases were purified and isolated to investigate their in vitro kinetic behavior with various C4 and C2 substrates. In particular, the monofunctional aldehyde dehydrogenase identified in FIG. 3 above (GI 150018649, also referred to as ALDH16) was purified (FIG. 4A and FIG. 4B) and the in vitro kinetic behavior of a purified Strep-ALDH16 protein with various C4 and C2 substrates was tested. The results of this analysis are presented in Table 4.

TABLE 4
In Vitro Kinetics of Aldehyde/Alcohol Dehydrogenases

| | Substrate | k_{cat} (s⁻¹) | K_{M} (μM) | k_{cat}/K_{M} (M⁻¹ s⁻¹) |
|---|---|---|---|---|
| AdhE2- | butyryl- | 1.2 ± 0.1 | 10 ± 1 | (1.1 ± 0.1) × 10⁵ |
| ALDH | CoA | | | |
| | acetyl- | 1.3 ± 0.1 | 100 ± 10 | (1.3 ± 0.2) × 10⁴ |
| | CoA | | | |
| AdhE2- | butyral- | 2.9 ± 0.1 | 4000 ± 400 | (7.0 ± 0.2) × 10² |
| ADH | dehyde | | | |
| | acetal- | 5.6 ± 0.1 | 4500 ± 300 | (1.2 ± 0.1) × 10³ |
| | dehyde | | | |
| ALDH16 | butyryl- | 0.11 ± 0.01 | 14 ± 2 | (7.3 ± 0.01) × 10³ |
| | CoA | | | |
| | acetyl- | 0.05 ± 0.01 | 454 ± 98 | (0.1 ± 0.01) × 10³ |
| | CoA | | | |

From Table 4, it is seen that the ALDH domain of AdhE2 displays a modest 8.5-fold preference for butyryl-CoA over acetyl-CoA. Furthermore, the K_{M} of 100±10 μM for acetyl-CoA is well below the physiological concentration of acetyl-CoA, thus enabling significant ethanol production. This is in contrast to one of the monofunctional aldehyde dehydrogenases characterized in this study, ALDH16, which exhibits a 73-fold preference for butyryl-CoA over acetyl-CoA and a 5-fold higher K_{M} for acetyl-CoA. The ADH domain of AdhE2 displays a slight 1.7-fold preference for acetaldehyde over butyraldehyde. However, this result is of little consequence because the overall specificity of any ALDH/ADH pair will be primarily dictated by the upstream ALDH. This enables a tailored ALDH with a given substrate specificity to be paired with a range of ADHs that may be relatively less discriminatory in substrate preference; the ALDH acts as a gatekeeper and only makes a preferred aldehyde intermediate available to an ADH.

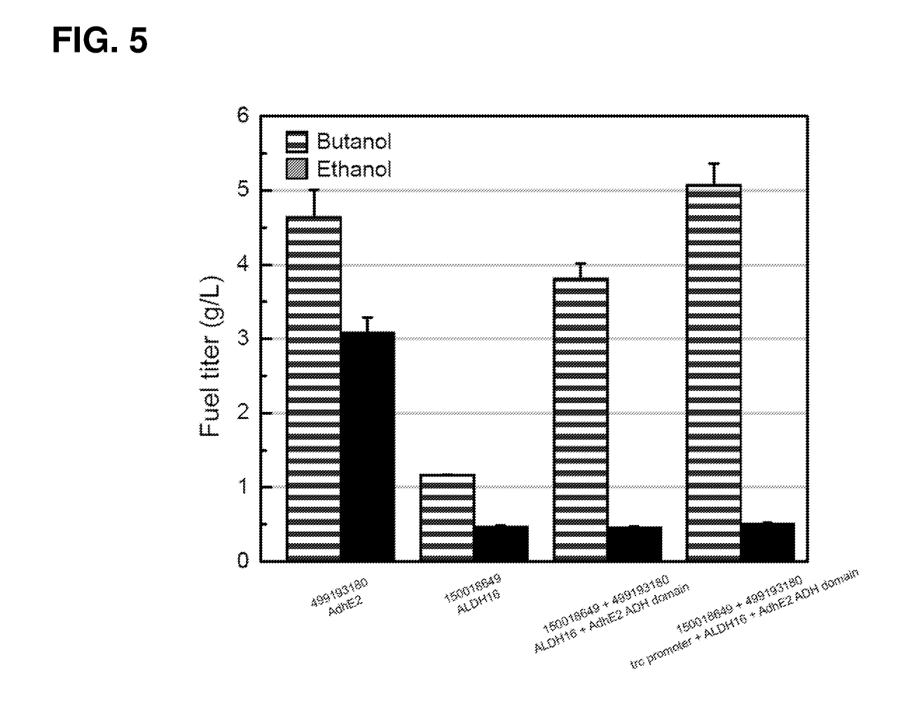

To demonstrate that a biosynthetic pathway incorporating split monofunctional aldehyde/alcohol dehydrogenases can act as a high-flux fermentation pathway, a more effective butanol production pathway in E. coli was constructed using ALDH16 as the monofunctional aldehyde dehydrogenase and the AdhE2-ADH domain as the monofunctional alcohol dehydrogenase (FIG. 5). The corresponding AdhE2-based pathway is capable of producing 4.5 g/L butanol and 3 g/L ethanol. Replacement of AdhE2 with only ALDH16 decreases production to 1 g/L butanol and 0.5 g/L ethanol, but inclusion of the AdhE2-ADH domain restores robust butanol production to nearly 4 g/L with no additional ethanol production. Improved gene expression in the pathway enables greater than 5 g/L butanol production while maintaining minimal ethanol production of 0.5 g/L (FIG. 5). This shows that a robust fermentation pathway can be developed with monofunctional aldehyde/alcohol dehydrogenases despite the release of a volatile and reactive aldehyde intermediate.

As discussed above, the initial butanol production pathway (adhE2) utilized a bifunctional aldhehyde/alcohol dehydrogenase that lacked substrate specificity and produced significant quantities of ethanol. However, upon discovering a monofunctional aldehyde dehydrogenase that was specific for C4 substrates (aldh16), this pathway was completed with a monofunctional alcohol dehydrogenase (aldh16.adh) and the expression of this new pathway architecture was improved (trc.aldh16.adh). The butanol production from this more efficient pathway exceeds that from the adhE2 pathway while decreasing ethanol production to a minimal level (FIG. 5), highlighting the unexpected result that a high-flux pathway can be constructed despite proceeding through a volatile and reactive aldehyde intermediate.

Further, a genetically engineered strain was produced where production of 8 g/L butanol was achieved with this more effective set of aldehyde/alcohol dehydrogenases. This particular strain contains the biosynthetic components to produce butanol and is further a quintuple knockout E. coli strain (ΔadhE ΔldhA ΔackA-pta ΔpoxB ΔfrdBC) with overexpression of the pyruvate dehydrogenase complex and grown under anaerobic conditions.

Further, to demonstrate that a biosynthetic pathway incorporating split monofunctional aldehyde/alcohol dehydrogenases can act as a high-flux fermentation pathway for multiple alcohols, pathways for the production of n-butanol, crotyl alcohol, or 1,3-butanediol were constructed. The specific enzymes used in the production of each specific alcohol are presented in FIG. 6A-FIG. 6C. The genes were synthesized and their codon usage optimized for expression in E. coli. The genes in the pathways include phaA (acetoacetyl-CoA thiolase from Ralstonia eutrophus, GI 498509665), hbd (3-hydroxybutyryl-CoA dehydrogenase from Aeromonas caviae, GI 499268602), crt (crotonase from Clostridium acetobutylicum, GI 15895969), ter (trans-enoyl-CoA reductase from Treponema denticola, GI 488758537), aldh (aldehyde dehydrogenase from various species), and adh (alcohol dehydrogenase from various species).

The results of alcohol production in E. coli expressing the various biosynthetic pathways depicted in FIG. 6A-FIG. 6C are presented in FIG. 7. E. coli were engineered to contain either the pathway in FIG. 6A for n-butanol production, the pathway in FIG. 6B for crotyl alcohol production, or the pathway in FIG. 6C for 1,3-butanediol production. Each cell line contained one of the various selected aldehyde dehydrogenases (See x-axis of FIG. 7). It was seen that different members of the aldehyde dehydrogenase family can be used for the production of butanol, 1,3-butanediol, and crotyl alcohol. Additionally, the stereochemistry of these products can be precisely controlled through the selection of enzymes upstream of the aldehyde dehydrogenase. Co-expression of these genes in E. coli and analysis of the pathway has shown that the enzymes are functional and capable of producing butanol, 1,3-butanediol, and crotyl alcohol. Each aldehyde dehydrogenase exhibits a preferred substrate and/or different product profile (FIG. 7), and substrate preference and/or product profile can be further modified through engineered recombination of the naturally present diversity within the collection. The first generation of genetically engineered hosts was capable of producing butanol at titers of 1.6 g/L, 1,3-butanediol at titers of 0.8 g/L, or crotyl alcohol at titers of 6 mg/L under anaerobic conditions.

In addition to exploring the diversity of different aldehyde dehydrogenases with respect to the production of various alcohols, the diversity of alcohol dehydrogenases was also explored, especially with respect to the production of 1,3-butanediol. Similarity networks were created between alcohol dehydrogenases and 1,3-propanediol dehydrogenases to identify potential alcohol dehydrogenases for production of 1,3-butanediol (FIG. 8). Sixteen alcohol dehydrogenases were selected from the resulting clusters for further functional analysis.

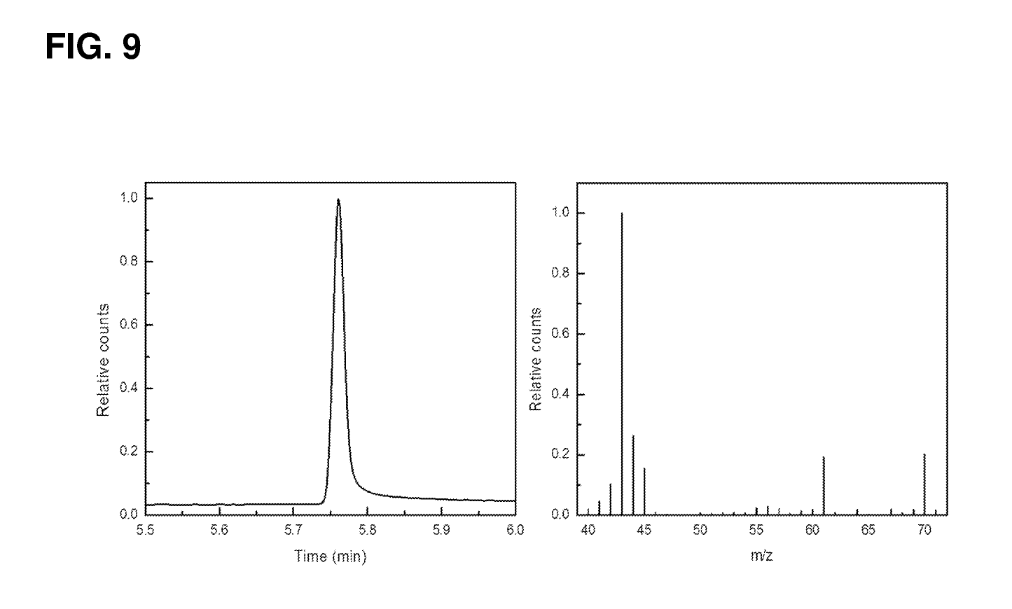

To investigate the impact of different alcohol dehydrogenases on 1,3-butanediol production, E. coli MC1.24 (DH1 ΔadhE ΔldhA Δack-pta ΔpoxB ΔfrdBC) was transformed with pT533-phaA.phaB and pCWO.trc-aldh16.adh 1-16, where each plasmid contained one of the sixteen adh genes identified from the network analysis (each E. coli line contained only one of the recombinant adh genes 1-16), and cultured anaerobically for 3 days. Culture supernatant was harvested and 1,3-butanediol titers were quantified by GC-MS. Retention time and fragmentation patterns from the culture supernatants were compared with a commercial authentic standard (FIG. 9). As was seen with the diverse set of aldehyde dehydrogenases, the expression of different alcohol dehydrogenases in the above E. coli lines engineered to produce 1,3-butanediol resulted in differential product yields amongst the different alcohol dehydrogenases (FIG. 10).

Production of 4-hydroxy-2-butanone

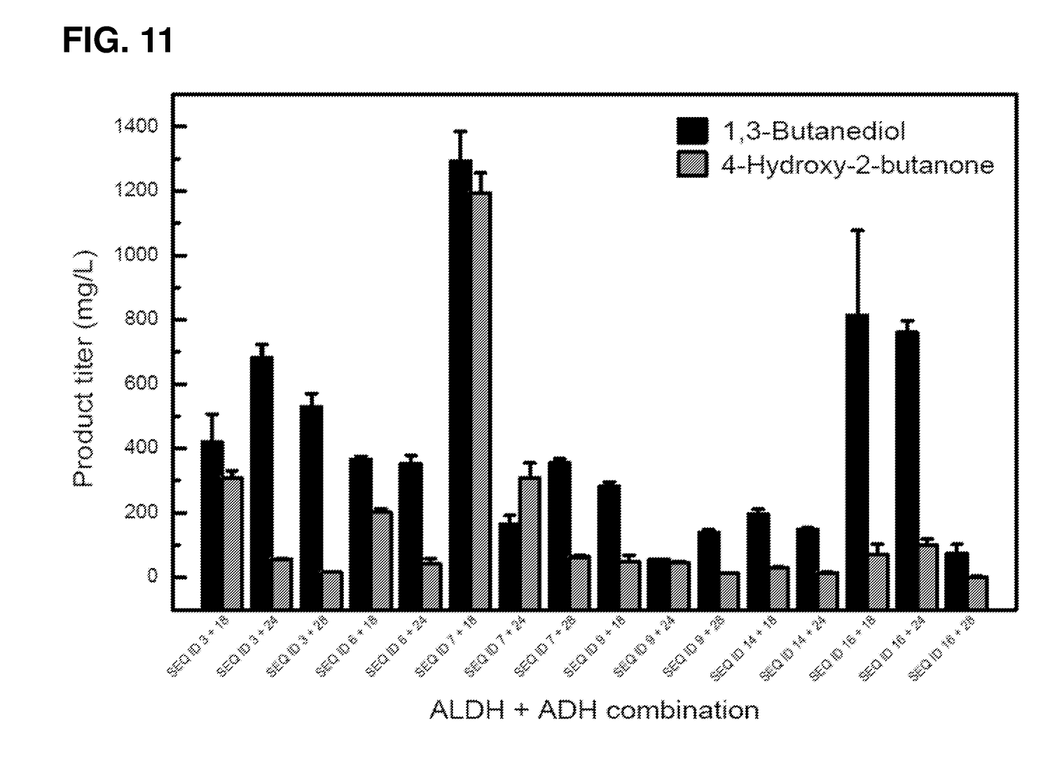

Surprisingly, some combinations of aldehyde and alcohol dehydrogenase also allowed for the production of an additional product, 4-hydroxy-2-butanone (FIG. 11). For example, an E. coli line containing the combination of ALDH7 (GI 187934965, SEQ ID NO: 7) and ADH2 (UniProt G5F136_9ACTN, SEQ ID NO: 18) produced nearly equivalent levels of 1,3-butanediol and 4-hydroxy-2-butanone of around 1.2 g/L. Production titers of 4-hydroxy-2-butanone were very high in this particular combination of aldehyde and alcohol dehydrogenases; E. coli lines containing ALDH7 with alternate ADHs, or ADH2 with alternate ALDHs did not accumulate 4-hydroxy-2-butanone at such high levels.

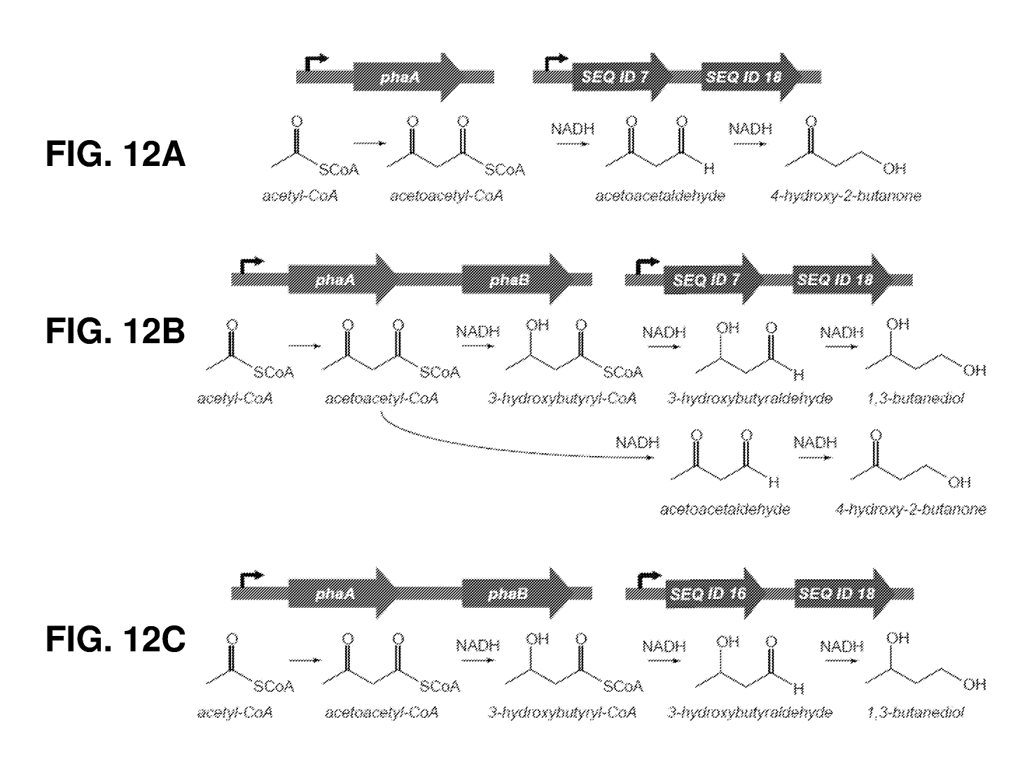

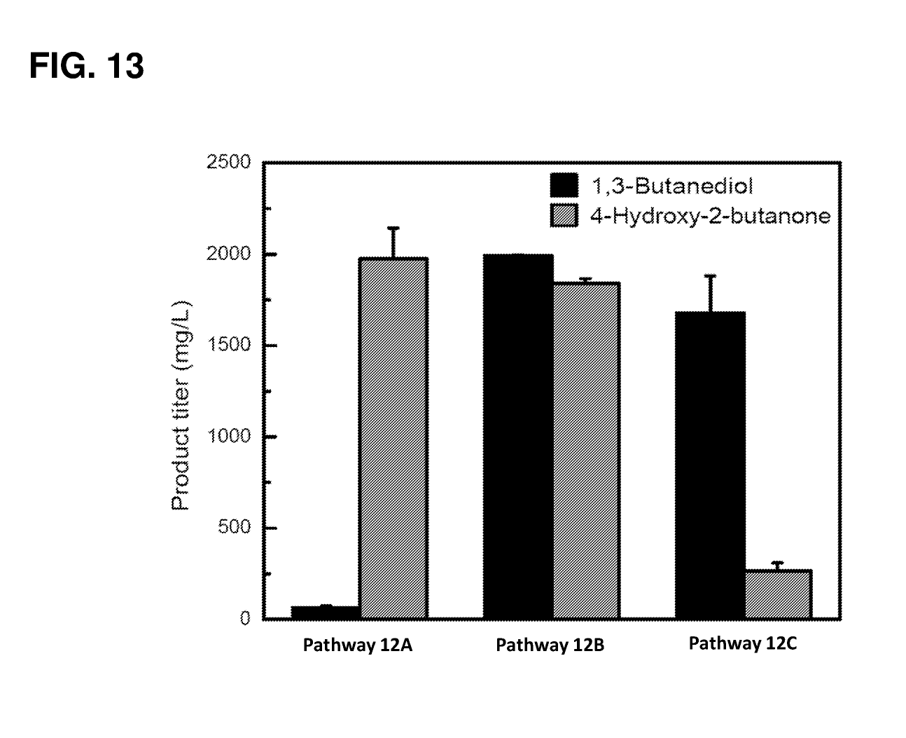

Further, in addition to evaluating the different combinations of aldehyde and alcohol dehydrogenases on the production of 4-hydroxy-2-butanone, different pathway variants engineered to contain various combinations of genetic components were also evaluated to determine the impact each variant pathway had on the production of 1,3-butanediol and/or 4-hydroxy-2-butanone (FIG. 12A-FIG. 12C). It was found that the pathway described in FIG. 12A allowed for the production of predominantly 4-hydroxy-2-butanone, the pathway described in FIG. 12B allowed for the production of both 1,3-butanediol and 4-hydroxy-2-butanone, and the pathway described in FIG. 12C allowed for the production of predominantly 1,3-butanediol (FIG. 13). Thus, the product ratio between 1,3-butanediol and 4-hydroxy-2-butanone can be tuned by transforming the E. coli cell line with pathway variants that allow for significant production of 4-hydroxy-2-butanone, 1,3-butanediol, or both of these compounds (FIG. 13).

Purification of ALDH3

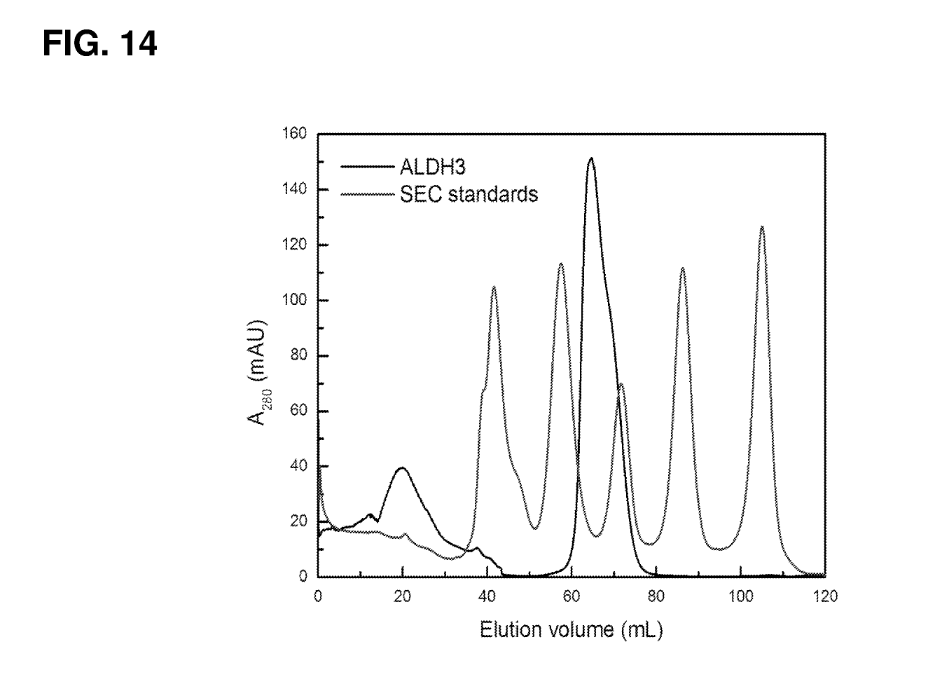

The aldehyde dehydrogenase ALDH3 (GI 31075383) was also recombinantly expressed and purified for experiments investigating the in vitro kinetic behavior of this protein with various C4 and C2 substrates. FIG. 14 illustrates a size-exclusion chromatogram of GA-ALDH3.

### Conclusion

Applicants have demonstrated that monofunctional aldehyde/alcohol dehydrogenases can be used for the production of various alcohols in host cells. Overall, this approach of using a combination of monofunctional aldehyde and alcohol dehydrogenases, rather than a single bifunctional enzyme, allows for greater exploration of sequence diversity and substrate specificity found in the monofunctional enzymes while preserving the high efficiency of transformation found in pathways involving bifunctional enzymes.

### Example 2: Analysis of Kinetic Properties of Monofunctional Aldehyde Dehydrogenase ALDH7

This Example describes the characterization of ALDH7 protein with respect to the in vitro kinetic behavior of this protein with various C4 and C2 substrates. This section also describes directed evolution of this protein to alter its substrate specificity or activity.

ALDH7 has NCBI GenInfo Identifier Number GI 187934965. This protein was recombinantly expressed and purified as described in Example 1.

To assess the kinetic behavior of ALDH7 with various substrates, the activity of ALDH7 protein will be measured in vitro by monitoring the oxidation of NADH at 340 nm at 30° C. The assay mixture (400 μL) will contain 100 μM NADH in 100 mM Tris 1 mM DTT pH 7.5. The reaction will be initiated by the addition of substrate. Substrates to be analyzed include acetyl-CoA and butyryl-CoA. Kinetic parameters (k_{cat}, K_{M}) will be determined by fitting the data using Microcal Origin to the equation: vₒ=vₘₐₓ [S]/(K_{M}+[5]), where v is the initial rate and [5] is the substrate concentration. Data will be reported as mean±s.e. (n=3) unless otherwise noted and standard error will be derived from the nonlinear curve fitting. Error in k_{cat}/K_{M} will be calculated by propagation of error from the individual kinetic parameters.

ALDH7 will also be subjected to directed evolution to alter its substrate specificity or activity. Applicants found that ALDH7 displays activity toward a range of substrates including acetoacetyl-CoA and 3-hydroxybutyryl-CoA. As was seen in Example 1, this broad substrate specificity enabled E. coli lines expressing ALDH7 to produce both 4-hydroxy-2-butanone and 1,3-butanediol respectively. Alteration of the substrate specificity could shift the product profile to only 4-hydroxy-2-butanone, only 1,3-butanediol, or variable mixtures of both compounds.

Directed evolution of ALDH7 will be pursued via DNA shuffling. SEQ ID NO: 1-16 are between 52% and 97% identical at the nucleic acid level. These sequences will be partially digested with DNaseI and reassembled via PCR to create chimeric fusion sequences containing fragments from multiple parental sequences. These chimeric sequences will be transformed into E. coli lines and screened for 4-hydroxy-2-butanone and 1,3-butanediol production according to the methods described in Example 1. Chimeric sequences displaying desirable properties can be used as parental sequences for additional rounds of DNA shuffling in an iterative fashion.

Directed evolution of ALDH7 will also be pursued via saturation mutagenesis. A structural homology model of ALDH7 will be built using the I-TASSER protein structure prediction webserver (http://zhanglab.ccmb.med.umich.edu/I-TASSER/) and used to investigate the active site of the protein. Residues surrounding the active site will be subjected to saturation mutagenesis, transformed into E. coli lines, and screened for 4-hydroxy-2-butanone and 1,3-butanediol production. Mutant sequences displaying desirable properties can be used as parental sequences for additional rounds of saturation mutagenesis in an iterative fashion.

### Example 3: Control of Hydroxybutanone and Butanediol Production Through Expression of a Monofunctional Secondary Alcohol Dehydrogenase

This Example describes how expression of a monofunctional secondary alcohol dehydrogenase can be used to control the production of 4-hydroxy-2-butanone and 1,3-butanediol in a pathway that can produce both of these products.

### Introduction

In Example 1, it was found that certain combinations of aldehyde and alcohol dehydrogenases in a biochemical pathway containing an acetoacetyl-CoA thiolase/synthase (e.g. phaA) and an acetoacetyl-CoA reductase (e.g. phaB) allowed for the production of both 1,3-butanediol and 4-hydroxy-2-butanone from acetyl-CoA starting material (See e.g. FIG. 12B). This result stemmed from the appearance of an unexpected peak in GC-MS quantification of butanediol production in a pathway modeled off of the pathway presented in FIG. 6C. It was found that 4-hydroxy-2-butanone was being produced as a significant side-product present in some cultures. Hydroxybutanone may be produced by reduction of an earlier pathway intermediate, acetoacetyl-CoA, by an ALDH, followed by subsequent reduction of acetoacetaldehyde by an ADH (See FIG. 12B). It was also described in Example 1 that the product ratio between 1,3-butanediol and 4-hydroxy-2-butanone can be tuned by transforming the E. coli cell line with pathway variants that allow for significant production of 4-hydroxy-2-butanone, 1,3-butanediol, or both of these compounds (FIG. 13).

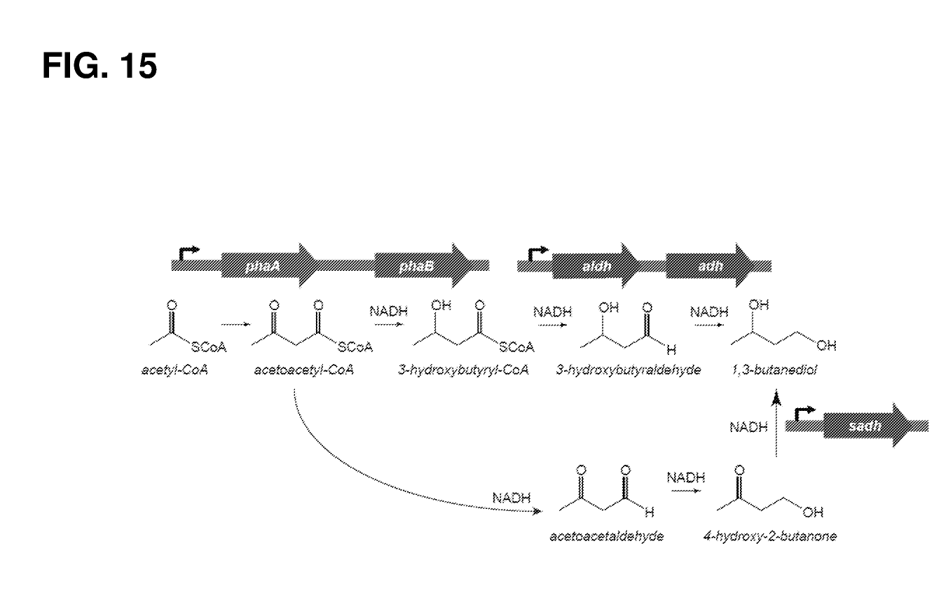

As an alternative strategy for altering the ratio of butanediol and hydroxybutanone that would not preclude off-pathway acetoacetaldehyde from conversion to butanediol, Applicant designed a pathway that contained a secondary alcohol dehydrogenase (SADH) such that this pathway would accept acetoacetaldehyde, reduce it to 4-hydroxy-2-butanone, and then further reduce it to 1,3-butanediol (FIG. 15). It was thought that the net product of this pathway would ultimately be butanediol, but some carbon would be channeled through 3-hydroxybutyraldehyde and some carbon would be channeled through acetoacetaldehyde. This Example describes how use of various secondary alcohol dehydrogenases allows for fine-tuning of the production of hydroxybutanone and butanediol.

### Materials and Methods

Unless otherwise described, applicable materials and methods as described in this Example are analogous to those described in Example 1.

### Results

To implement the pathway described in FIG. 15, the biochemical literature was thoroughly surveyed to identify secondary alcohol dehydrogenases (SADHs) either reported to reduce 4-hydroxy-2-butanone to 1,3-butanediol or reported to have broad specificity for similar substrates. A substantial number of these enzymes have been reported in bacteria, yeast, and parasitic protozoa. These enzymes are generally classified as zinc or iron-alcohol dehydrogenases and maximum percent identity within the sequences represented here range from 27-76% (Table 5).

TABLE 5
Identification of secondary alcohol dehydrogenases
for reduction of hydroxybutanone to butanediol

| | Gene | | SEQ |
|---|---|---|---|
| Organism | Name | Accession | ID NO. |
| Pichia kudriavzevii | SADH1 | KGK36767.1 | 250 |
| Pyrococcus furiosus | SADH2 | WP_011011186.1 | 251 |
| DSM 3638 | | | |
| Cupriavidus necator | SADH3 | WP_011614641.1 | 252 |
| Thermoanaerobacter | SADH4 | P14941.1 | 253 |
| brockii | | | |
| Clostridium beijerinckii | SADH5 | AAA23199.2 | 254 |
| Kluyveromyces lactis | SADH6 | XP_455102.1 | 255 |
| NRRL Y-1140 | | | |
| Phytomonas sp. ADU-2003 | SADH7 | AAP39869.1 | 256 |
| Ralstonia eutropha H16 | SADH8 | Q0KDL6.1 | 257 |
| Trichomonas vaginalis G3 | SADH9 | XP_001580601.1 | 258 |
| Pseudomonas fluorescens | SADH10 | AJP52792.1 | 259 |
| Lactococcus lactis | SADH11 | WP_011835462.1 | 260 |
| Saccharomyces cerevisiae | SADH12 | AAC04974.1 | 261 |
| Escherichia coli | SADH13 | WP_000374004.1 | 262 |
| Zygoascus ofunaensis | SADH14 | BAD32689.1 | 263 |
| Candida parapsilosis | SADH15-2 | BAA24528.1 | 264 |
| Cyberlindnera jadinii | SADH16 | BAN45671.1 | 265 |
| Rhodococcus ruber | SADH17 | CAD36475.1 | 266 |

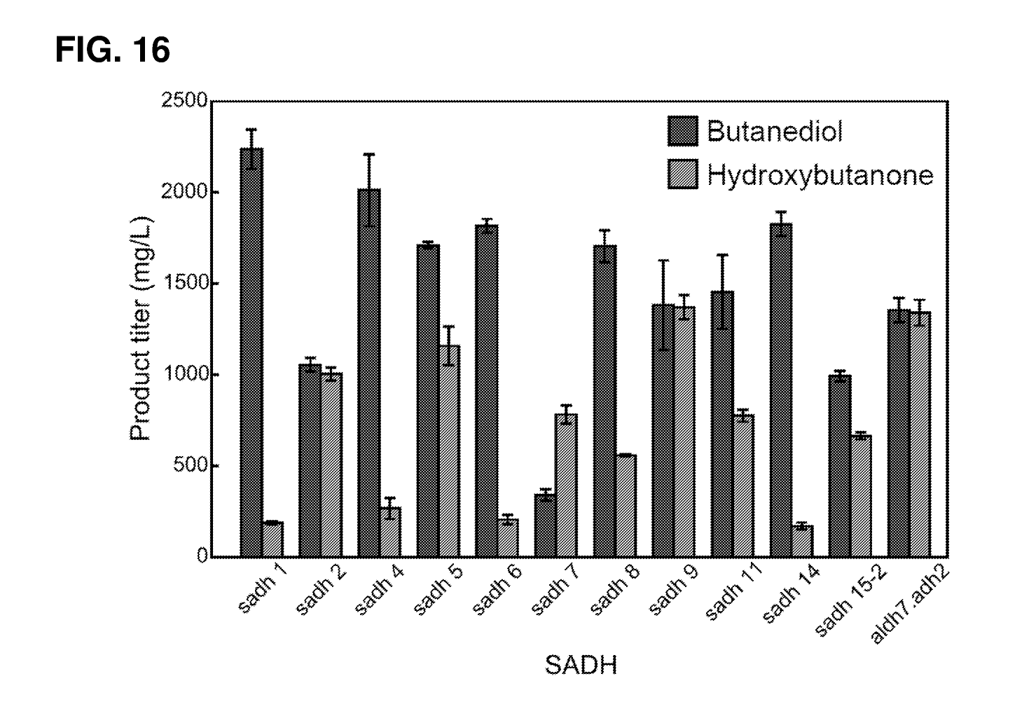

The identified SADHs in Table 5 were cloned into E. coli in pathways with aldh7 and adh2 (which consistently produced an even mixture of butanediol and hydroxybutanone, See FIG. 13), transformed E. coli were cultured, and metabolite production was quantified. It was found that many SADHs shifted the product profile compared to the aldh7-adh2 control. At least four SADHs enabled butanediol production of 2 g/L with hydroxybutanone production limited to 250 mg/L or less (FIG. 16). This pathway design appears preferable to enforcing specificity through an ADH that will not accept acetoacetaldehyde; acetoacetaldehyde need not be a dead end product and can still be channeled to butanediol production.

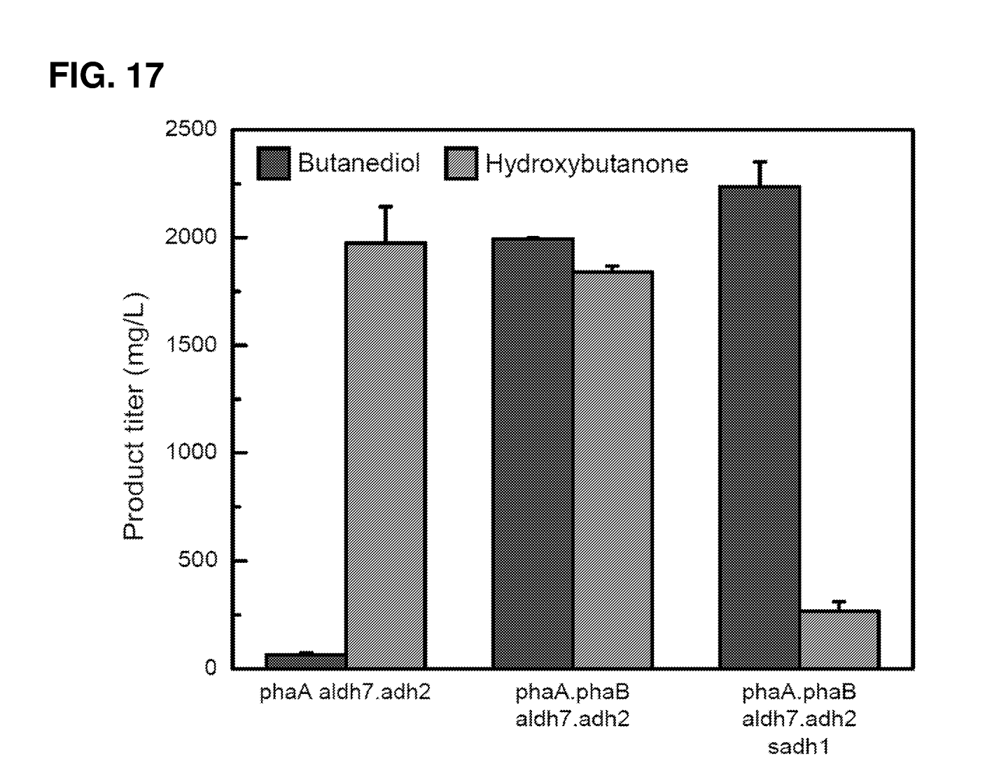

Extensive screening of candidate ALDHs, SADHs, and pathway improvements enabled production of pathways that exhibit tight control of the butanediol:hydroxybutanone product profile (FIG. 16 and FIG. 17). Maximum hydroxybutanone production was achieved with a pathway that did not express an acetoacetyl-CoA reductase and thus can only supply acetoacetyl-CoA to aldh7-adh2. An even mixture of products was achieved when an acetoacetyl-CoA reductase was added, thus allowing aldh7-adh2 to reduce both acetoacetyl-CoA and 3-hydroxybutyrl-CoA. Finally, maximum butanediol titer was achieved when the pathway was equipped with sadh1, which yields a two-tier pathway where half of the flux proceeds through 3-hydroxybutyryl-CoA to butanediol and half of the flux proceeds through hydroxybutanone to butanediol. Thus, in addition to use of more selective ALDHs which do not reduce acetoacetyl-CoA, this data demonstrates that 4-hydroxy-2-butanone production can be limited by expression of a secondary alcohol dehydrogenase (SADH) which reduces any accumulated 4-hydroxy-2-butanone to 1,3-butanediol.

### CONCLUSION

This Example has demonstrated an expansion of butanediol and hydroxybutanone production pathways with the addition of a secondary alcohol dehydrogenase that can reduce 4-hydroxy-2-butanone to 1,3-butanediol. This addition allows for recapturing off-pathway carbon diverted to hydroxybutanone should butanediol be the desired product, as well as producing a finely tuned product profile control should a mixture of products be desired. This mixture of products may be useful for polymer precursor production. The ability to deliver a product profile through balancing expression level of the enzymes expressed in this pathway affords a great deal of control, and opens the door to applications where tunable product profile is desired, such as catalytic upgrading to longer-chain compounds.

1. A recombinant host cell that facilitates the production of an alcohol from an acyl-CoA, wherein the host cell comprises:
a) a first nucleic acid which encodes a polypeptide involved in the stepwise conversion of an acyl-CoA to a substrate for a monofunctional aldehyde dehydrogenase;
b) a second nucleic acid which encodes a monofunctional aldehyde dehydrogenase; and
c) a third nucleic acid which encodes a monofunctional alcohol dehydrogenase;
wherein at least one nucleic acid selected from the group consisting of the first nucleic acid, the second nucleic acid, and the third nucleic acid is a recombinant nucleic acid.

2. The host cell of claim 1, wherein the host cell is E. coli.

3. The host cell of claim 1, wherein at least two nucleic acids selected from the group consisting of the first nucleic acid, the second nucleic acid, and the third nucleic acid are separate nucleic acids.

4. The host cell of claim 1, wherein the recombinant nucleic acid encodes a polypeptide selected from an acetoacetyl-CoA thiolase, a 3-hydroxybutyryl-CoA dehydrogenase, a crotonase, a trans-enoyl-CoA reductase, a monofunctional aldehyde dehydrogenase, a monofunctional alcohol dehydrogenase or any combination thereof.

5. The host cell of claim 4, wherein the acetoacetyl-CoA thiolase has at least 80% amino acid identity to SEQ ID NO: 33, the 3-hydroxybutyryl-CoA dehydrogenase has at least 80% amino acid identity to SEQ ID NO: 34, the crotonase has at least 80% amino acid identity to SEQ ID NO: 36, the trans-enoyl-CoA reductase has at least 80% amino acid identity to SEQ ID NO: 37, or any combination thereof.

6. The host cell of claim 4, wherein the monofunctional aldehyde dehydrogenase has at least 80% amino acid identity to SEQ ID NO: 16, the monofunctional alcohol dehydrogenase has at least 80% amino acid identity to SEQ ID NO: 17, or the monofunctional aldehyde dehydrogenase has at least 80% amino acid identity to SEQ ID NO: 16 and the monofunctional alcohol dehydrogenase has at least 80% amino acid identity to SEQ ID NO: 17.

7. The host cell of claim 1, wherein the acyl-CoA is acetyl-CoA.

8. The host cell of claim 1, wherein the alcohol is selected from n-butanol, crotyl alcohol, 1,3-butanediol, 4-hydroxy-2-butanone, or any combination thereof.

9. The host cell of claim 1, wherein the host cell exhibits reduced activity of one or more polypeptides selected from the group consisting of adhE, ldhA, ack-pta, poxB, and frdBC, or homologs thereof, as compared to a corresponding control cell.

10. The host cell of claim 9, wherein the host cell comprises knockout mutations in adhE, ldhA, ack-pta, poxB, and frdBC, or homologs thereof.

11. The host cell of claim 1, wherein the host cell further comprises a monofunctional secondary alcohol dehydrogenase.

12. The host cell of claim 11, wherein the monofunctional secondary alcohol dehydrogenase has at least 80% amino acid identity to SEQ ID NO: 250.

13. A recombinant host cell for the production of n-butanol, the host cell comprising:
a) a nucleic acid encoding an acetoacetyl-CoA thiolase capable of catalyzing the conversion of acetyl-CoA to acetoacetyl-CoA;
b) a nucleic acid encoding a 3-hydroxybutyryl-CoA dehydrogenase capable of catalyzing the conversion of acetoacetyl-CoA to 3-hydroxybutyryl-CoA;
c) a nucleic acid encoding a crotonase capable of catalyzing the conversion of 3-hydroxybutyryl-CoA to crotonyl-CoA;
d) a nucleic acid encoding a trans-enoyl-CoA reductase capable of catalyzing the conversion of crotonyl-CoA to butyryl-CoA;
e) a nucleic acid encoding a monofunctional aldehyde dehydrogenase capable of catalyzing the conversion of butyryl-CoA to butyraldehyde;
f) a nucleic acid encoding a monofunctional alcohol dehydrogenase capable of catalyzing the conversion of butyraldehyde to n-butanol;
wherein one or more of the nucleic acids are recombinant, and wherein the host cell is capable of producing at least 10-fold more n-butanol than ethanol.

14. A recombinant host cell for the production of crotyl alcohol, the host cell comprising:
a) a nucleic acid encoding an acetoacetyl-CoA thiolase capable of catalyzing the conversion of acetyl-CoA to acetoacetyl-CoA;
b) a nucleic acid encoding a 3-hydroxybutyryl-CoA dehydrogenase capable of catalyzing the conversion of acetoacetyl-CoA to 3-hydroxybutyryl-CoA;
c) a nucleic acid encoding a crotonase capable of catalyzing the conversion of 3-hydroxybutyryl-CoA to crotonyl-CoA;
d) a nucleic acid encoding a monofunctional aldehyde dehydrogenase capable of catalyzing the conversion of crotonyl-CoA to crotonaldehyde;
e) a nucleic acid encoding a monofunctional alcohol dehydrogenase capable of catalyzing the conversion of crotonaldehyde to crotyl alcohol;
wherein one or more of the nucleic acids are recombinant.

15. A recombinant host cell for the production of 1,3-butanediol, the host cell comprising:
a) a nucleic acid encoding an acetoacetyl-CoA thiolase capable of catalyzing the conversion of acetyl-CoA to acetoacetyl-CoA;
b) a nucleic acid encoding a 3-hydroxybutyryl-CoA dehydrogenase capable of catalyzing the conversion of acetoacetyl-CoA to 3-hydroxybutyryl-CoA;
c) a nucleic acid encoding a monofunctional aldehyde dehydrogenase capable of catalyzing the conversion of 3-hydroxybutyryl-CoA to 3-hydroxybutyraldehyde;
d) a nucleic acid encoding a monofunctional alcohol dehydrogenase capable of catalyzing the conversion of 3-hydroxybutyraldehyde to 1,3-butanediol;
wherein one or more of the nucleic acids are recombinant.

16. A recombinant host cell for the production of 4-hydroxy-2-butanone, the host cell comprising:
a) a nucleic acid encoding an acetoacetyl-CoA thiolase capable of catalyzing the conversion of acetyl-CoA to acetoacetyl-CoA;
b) a nucleic acid encoding a monofunctional aldehyde dehydrogenase;
c) a nucleic acid encoding a monofunctional alcohol dehydrogenase;
wherein one or more of the nucleic acids are recombinant.

17. A recombinant host cell for the production of one or more C4 alcohols, the host cell comprising:
a) a nucleic acid encoding an acetoacetyl-CoA thiolase;
b) a nucleic acid encoding a 3-hydroxybutyryl-CoA dehydrogenase;
c) a nucleic acid encoding a crotonase;
d) a nucleic acid encoding a trans-enoyl-CoA reductase;
e) a nucleic acid encoding a monofunctional aldehyde dehydrogenase;
f) a nucleic acid encoding a monofunctional alcohol dehydrogenase;
wherein one or more of the nucleic acids are recombinant,
and wherein the host cell is capable of producing a C4 alcohol at concentrations that are at least 10-fold higher than the concentration of ethanol produced by the host cell.

18. The host cell of claim 17, wherein the C4 alcohol is selected from n-butanol, crotyl alcohol, 1,3-butanediol, 4-hydroxy-2-butanone, or any combination thereof.

19. A method of producing an alcohol from an acyl-CoA, the method comprising:
a) providing the recombinant host cell of claim 1;
b) culturing the recombinant host cell in a culture medium comprising a suitable carbon source such that the host cell produces an alcohol.

20. The method of claim 19, further comprising a step of substantially purifying the alcohol from the culture medium.

21. A method of producing n-butanol, the method comprising:
a) providing the recombinant host cell of claim 13;
b) culturing the recombinant host cell in a culture medium comprising a suitable carbon source such that the host cell produces n-butanol,
wherein the host cell produces at least 10-fold more n-butanol than ethanol.

22. The method of claim 21, further comprising a step of substantially purifying n-butanol from the culture medium.

23. A method of producing crotyl alcohol, the method comprising:
a) providing the recombinant host cell of claim 14;
b) culturing the recombinant host cell in a culture medium comprising a suitable carbon source such that the host cell produces crotyl alcohol.

24. The method of claim 23, further comprising a step of substantially purifying crotyl alcohol from the culture medium.

25. A method of producing 1,3-butanediol, the method comprising:
a) providing the recombinant host cell of claim 15;
b) culturing the recombinant host cell in a culture medium comprising a suitable carbon source such that the host cell produces 1,3-butanediol.

26. The method of claim 25, further comprising a step of substantially purifying 1,3-butanediol from the culture medium.

27. A method of producing 4-hydroxy-2-butanone, the method comprising:
a) providing the recombinant host cell of claim 16;
b) culturing the recombinant host cell in a culture medium comprising a suitable carbon source such that the host cell produces 4-hydroxy-2-butanone.

28. The method of claim 27, further comprising a step of substantially purifying 4-hydroxy-2-butanone from the culture medium.

29. A method of producing one or more C4 alcohols, the method comprising:
a) providing the recombinant host cell of claim 17;
b) culturing the recombinant host cell in a culture medium comprising a suitable carbon source such that the host cell produces a C4 alcohol,
wherein the host cell produces the C4 alcohol at concentrations that are at least 10-fold higher than the concentration of ethanol produced by the host cell.

30. The method of claim 29, further comprising a step of substantially purifying a C4 alcohol from the culture medium.

31. The method of claim 29, wherein the C4 alcohol is selected from the group consisting of n-butanol, crotyl alcohol, 1,3-butanediol, and 4-hydroxy-2-butanone.

32. A recombinant polypeptide comprising an amino acid sequence selected from the group consisting of SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, and SEQ ID NO: 154.

33. A recombinant nucleic acid comprising a nucleotide sequence selected from the group consisting of SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, and SEQ ID NO: 249.

34. A recombinant host cell comprising the recombinant polypeptide of claim 32.

35. An expression vector comprising a recombinant nucleic acid of claim 33.

36. A recombinant host cell comprising the recombinant nucleic acid of claim 33.
